(19) United States
(12) Patent Application Publication
(10) Pub. No.: US 2023/0250480 A1
(43) Pub. Date: Aug. 10, 2023

(54) BIOFLUID-BASED PROTEIN AND MIRNA BIOMARKERS FOR NEONATAL HYPOXIC-ISCHEMIC ENCEPHALOPATHY

(71) Applicant: UNIVERSITY OF FLORIDA RESEARCH FOUNDATION, INCORPORATED, Gainesville, FL (US)

(72) Inventors: Kevin Ka W. WANG, Gainesville, FL (US); Zhihui YANG, Gainesville, FL (US); Michael D. WEISS, Gainesville, FL (US)

(21) Appl. No.: 18/015,169

(22) PCT Filed: Jul. 9, 2021
(86) PCT No.: PCT/US2021/041101

§ 371 Date: Jan. 9, 2023

Related U.S. Application Data
(60) Provisional application No. 63/050,048, filed on Jul. 9, 2020.

(51) Int. Cl. C12Q 1/6883; G01N 33/573; G01N 33/68

(52) U.S. Cl. CPC C12Q 1/6883; G01N 33/573; G01N 33/6893; C12Q 2600/158; C12Q 2600/178; G01N 2800/2871; G01N 2333/916

(57) ABSTRACT

The invention relates to a combination or panel of HIE-relevant protein and/or microRNA (miRNA) biomarkers that are released from injured tissues into biofluids such as blood in HIE, and their use as markers for detection of HIE. A selected panel of blood-based protein and/or miRNA HIE biomarkers that are measured at more than one time interval can aid in the diagnosis of HIE severity, and to determine the prognosis of poor versus good cognitive or overall patient outcome.

### BACKGROUND

### 1. Field of the Invention

The invention relates to the field of medicine, and in particular to methods for diagnosis, prognosis, and management of neonatal encephalopathy (NE), hypoxic-ischemic encephalopathy (HIE) and other related conditions that produce a risk to limb, organ, or life. In particular, the invention relates to a combination or panel of NE-relevant protein and/or microRNA (miRNA) biomarkers that are released from injured tissues into biofluids such as blood in NE, and their use as markers for detection of NE. A selected panel of blood-based protein and/or miRNA NE biomarkers that are measured at more than one time interval can aid in the diagnosis of NE severity, and to determine the prognosis of poor versus good cognitive or overall patient outcome.

### 2. Background

NE is a significant cause of morbidity and mortality in neonates. The incidence of NE ranges from 1 to 8 per 1000 live births in developed countries to as high as 26 per 1000 live births in underdeveloped countries. Of neonates with NE that survive, up to 25% have permanent neurodevelopmental handicaps in the form of cerebral palsy (CP), intellectual disabilities, learning disabilities, or epilepsy. NE also has a significant financial impact on the health care system. For example, in Florida, the total cost for initial hospitalization is $161,000 per NE patient admitted, not taking into account the continuing life-long costs. The Florida Birth-Related Neurological Injury Compensation Plan (NICA), pays about $3 million per claim to assist families of NE neonates.

Currently, therapy for NE neonates is limited to supportive care. Therapeutic hypothermia improves the neurodevelopmental outcome in infants with moderate NE. However, more than 7 out of 8 treated infants do not benefit from hypothermia, highlighting the need for treatment stratification according to injury severity. To be effective, hypothermia should be initiated as soon as possible and no later than six hours after the initial insult. Unfortunately, though, it is not possible, using standard clinical criteria, to accurately identify those neonates who will benefit from hypothermia versus non-responders. This is especially true after treatment has commenced due to the sedatives administered and the effects of the hypothermia treatment itself.

Current monitoring and evaluation of NE, outcome prediction, and efficacy of hypothermia treatment rely on a combination of a neurological examination, ultrasound, magnetic resonance imaging (MRI), and electroencephalography (EEG). However, these methods do not adequately identify hypothermia non-responders. MRI requires transport of the neonate and a scan that takes about 40-45 minutes, which is not suitable for neonates in unstable condition. Moreover, the amplitude integrated EEG (aEEG), a common bedside monitoring technique currently used in these patients, can be adversely affected by hypothermia itself and the neonates may or may not appear to improve until re-warming. Consequently, there is a need in the art for a simple, inexpensive, non-invasive, rapid biochemical test to identify suitable neonatal candidates for therapeutic hypothermia, to distinguish hypothermia treatment responders from non-responders, and to assess outcome for NE.

### SUMMARY

The invention discussed herein relates to certain biomarkers in combination that can be detected in biofluids of NE patients, including but not limited to Glial fibrillary acidic protein (GFAP), ubiquitin C-terminal hydrolase L1 (UCHL-1), Tau protein, Neurofilament light chain (NF-L) and a panel of 39 miRNA (Table 3 and Table 4). These biomarker peptides are markers of brain cells, axonal and blood brain barrier integrity which can be used to diagnose the condition and to assess and predict cognitive, speech and motor performance and outcomes in acute presentations of hypoxia & ischemia. In certain embodiments, methods according to the invention can be used to identify and analyze changes in biomarker levels which are associated with hypoxic-ischemic encephalopathy (HIE), particularly in neonatal human patients. Differential values of selected biomarkers can provide information concerning whether a particular patient will respond to hypothermia treatment, and information on the severity and prognosis of NE in that patient. Biomarker analysis (including relative levels of the biomarkers and ratios of the biomarkers) can provide useful tools for diagnosis, prognosis and management of neonatal NE. Biomarkers which are contemplated for use in the invention include, but are not limited to, glial fibrillary acidic protein (GFAP), ubiquitin C-terminal hydrolase (UCH-L1), Tau, and neurofilament light chain (NF-L) and combinations thereof. Preferably the invention includes use of all 4 of these protein biomarkers in a panel.

The invention also includes, in certain embodiments, microRNA (miRNA) biomarkers, which include, but are not limited to hsa-mir-145-5p, hsa-mir-16-5p, hsa-mir-15a-5p, hsa-mir-17-5p, hsa-let-7g-5p, hsa-mir-214-3p, hsa-mir-338-3p, hsa-mir-132-3p, hsa-mir-23a-3p, hsa-mir-26b-5p and hsa-mir-146a-5p.

Therefore, the invention provides:

### BRIEF DESCRIPTION OF THE DRAWINGS

FIG. 1 provides a profile of neuroprotein biomarkers for HIE, including GFAP (FIG. 1A), UCH-L1 (FIG. 1B), NF-L (FIG. 1C), and Tau (FIG. 1D).

FIG. 2 shows blood concentrations at the indicated times for GFAP (FIG. 2A), UCH-L1 (FIG. 2B), NF-L (FIG. 2C), and Tau (FIG. 2D).

FIG. 3 shows blood level of GFAP (FIG. 3A, FIG. 3B, and FIG. 3C), UCH-L1 (FIG. 3D, FIG. 3E, and FIG. 3F), NF-L (FIG. 3G, FIG. 3H, and FIG. 3I), and Tau (FIG. 3J, FIG. 3K, and FIG. 3L) correlated with scores for basal ganglia MRI (FIG. 3A, FIG. 3D, FIG. 3G, and FIG. 3J), watershed MRI (FIG. 3B, FIG. 3E, FIG. 3H, and FIG. 3K), and thalamus/basal ganglia/cortex MRI (FIG. 3C, FIG. 3F, FIG. 3I, and FIG. 3L).

FIG. 4 shows correlation of SARNAT score and protein biomarkers GFAP (FIG. 4A), UCH-L1 (FIG. 4B), Tau (FIG. 4C), and NF-L (FIG. 4D).

FIG. 5 shows protein biomarker levels GFAP (FIG. 5A, FIG. 5B, and FIG. 5C), UCH-L1 (FIG. 5D, FIG. 5E, and FIG. 5F), NF-L (FIG. 5G, FIG. 5H, and FIG. 5I), and Tau (FIG. 5J. FIG. 5K, and FIG. 5L) in blood at different time intervals, correlated to other HIE assessment tools pH (FIG. 5A, FIG. 5D, FIG. 5G, and FIG. 5J), lactate (FIG. 5B, FIG. 5E, FIG. 5H, and FIG. 5K), and sentinel event (FIG. 5C, FIG. 5F, FIG. 5I, and FIG. 5L).

FIG. 6 shows protein biomarker levels GFAP (FIG. 6A, FIG. 6B, and FIG. 6C), UCH-L1 (FIG. 6D, FIG. 6E, and FIG. 6F), NF-L (FIG. 6G, FIG. 6H, and FIG. 6I), and Tau (FIG. 5J. FIG. 6K, and FIG. 6L) in blood at different time intervals, correlated to Bayley Outcome scores for cognitive function (FIG. 6A, FIG. 6D, FIG. 6G, and FIG. 6J), language (FIG. 6B, FIG. 6E, FIG. 6H, and FIG. 6K), and motor function (FIG. 6C, FIG. 6F, FIG. 6I, and FIG. 6L).

FIG. 7 shows the biomarker trajectory for GFAP (FIG. 7A), NF-L (FIG. 7B), Tau (FIG. 7C), UCH-L1 (FIG. 7D), and for all four of these markers (FIG. 7E).

FIG. 8 shows the Bayler score for cognitive function (FIG. 8A), language function (FIG. 8B), and motor function (FIG. 8C) for class 1 and class 2 patients.

FIG. 9 shows human miRNA serum levels in HIE. FIG. 9A is a high baseline miRNA levels set and FIG. 9B is low baseline miRNA levels set.

FIG. 10 presents the indicated human miRNA serum levels in patients with high baseline levels and altered levels in HIE (FIG. 10A), medium baseline levels and altered levels in HIE (FIG. 10B), and low baseline levels and altered levels in HIE (FIG. 10C).

FIGS. 11A and 11B are a flow charts showing the use of point-of-care bedside HIE biomarker tests.

FIG. 12 is a schematic of a point of care diagnostic device embodiment.

FIG. 13 are graphs showing the temporal profile of serum levels of 4 neuroprotein biomarkers in NE cohort as compared to non NE controls. GFAP (1A), UCH-L1 (1B), NFL (1C) and Tau (1D) serum concentrations in heathy controls compared with NE (median and interquartile range are shown). The neonates with moderate to severe NE are represented at various sampling time points. Compared with healthy controls (#P<0.05, ##P<0.01, ###P<0.001).

FIG. 14 are graphs showing neuroprotein biomarker concentrations compared to MRI scores. 2A (top panel): MRI injury score for basal ganglia. 2B (mid panel): MRI injury score for watershed region. 2C (bottom panel): MRI injury score for basal ganglia/watershed regions. Infants had a MRI scan at 3 days of life following rewarming. The scores of lesion at basal ganglia, watershed, thalamus and cortex were calculated. * p<0.05. ** p<0.01, the comparison was between the neonates without injury or mild injury (MRI score=0-2) and those with moderate and severe injury (MRI score=3-5) (n=40).

FIG. 15 are graphs with the area under ROC curve performance using biomarker concentrations at 12h for outcome at 18-24 months follow-up. Bayley score for cognitive function (3A, left panel), language function (3B, mid panel) and motor function (3C, right panel).

FIG. 16 are graphs showing trajectory analysis of neurological outcomes at 18-24 months follow-up. Group trajectory is indicated by the solid line; 95% confidence intervals are indicated by dotted lines. Four markers HO-standardized score based latent trajectories. Temporal biomarker levels are standardized with HO levels (as 100). 4A, two-group biomarker trajectories profiles using concentration change from baseline. The y-axis represents the natural log back-transformed concentration ratio change from baseline. Legends indicate the number (%) of participants in each trajectory group. 4B, all patients in the high-trajectory group (class 2) belong to the poor neurologic outcome group, while 90% low-trajectory patients (class 1) belong to the good cognitive function group. 85% patients in low trajectory group have good outcome in term of language function. 4C, logistic Regression modelling predicting NE outcomes using biomarker trajectory groups. Odds ratio are expressed using Group 1 as the reference group.

FIG. 17 are graphs showing biomarker concentrations and correlation with developmental outcome (Bayley III scores). Neonates had Bayley exam performed between 18-24 months of age. The infants were classified as a good outcome in a domain if they Bayley score in that domain ≥85, while poor outcome is <85 A (top panel): Bayley III score for cognitive function. B (mid panel): Bayley III score for language function. C (bottom panel): Bayley III score for motor function. * The comparison was between the neonates with good outcome and those with poor outcome. *p<0.05, ** p<0.01. *** p<0.01.

FIG. 18 are graphs showing a HO-standardized score based latent trajectory classes show significantly different levels of the respective marker at H24, H 48 and H96. Class 2 members have high trajectory, while Class 1 members have lower trajectory. Serial serum concentrations of the four markers from patients within for the two trajectory classes were shown as separate box and whiskers at each time point *, **, *** p<0.05, <0.01, <0.001, respectively.

FIG. 19 are graphs showing a comparison of HIE Biomarkers GFAP, NFL, Tau, UCHL-1 among Control, mild HIE and moderate-severe HIE. Compared to Control group, serum concentration of GFAP was higher in mild HIE and moderate-severe HIE group (p<0.01 and p<0.001, respectively). The concentrations of NFL, Tau and UCHL-1 were increased in the neonates with moderate-severe HIE compared to the control neonates (p<0.05). No concentration differences were noted between NFL, UCH-L1 and Tau between controls and neonates with low cord pH with/without mild HIE at 0-6 hours of life.

FIG. 20 are graphs showing a comparison of neuroprotein biomarkers between controls, mild HIE and moderate to severe HIE. GFAP (A), NFL (B), Tau (C) and UCHL1 (D) serum concentrations in heathy controls, neonates with mild HIE and neonates with moderate to severe HIE neonates undergoing hypothermia treatment. Compared with healthy controls * P<0.05, ** P<0.01, ***P<0.001; Compared with mild HIE #p<0.05.

FIG. 21 are graphs showing serum concentrations of GFAP, NFL, Tau and UCH-L1 in neonates with a pH≤7 compared to a pH>7. GFAP (A), NFL (B), Tau (C) and UCHL1 (D) serum concentrations were higher in neonates with a pH≤7 compared to neonates with a pH>7 (* P<0.05, ** P<0.01)(Mean±STD).

FIG. 22 are graphs showing serum concentrations of GFAP, NFL, Tau and UCH-L1 in neonates with and without a sentinel event. GFAP (A), NFL (B), Tau (C) and UCHL1 (D) serum concentrations in neonates with and without a sentinel event (Mean±STD).

FIG. 23 are graphs showing serum concentrations of GFAP, NFL, Tau and UCH-L1 in neonates with a normal neurologic exam compared to those with mild HIE. GFAP (A), NFL (B), Tau (C) and UCHL1 (D) serum concentrations in neonates with a normal neurologic exam compared to those with mild HIE (Mean±STD).

### DETAILED DESCRIPTION

### 1. Definitions

Unless defined otherwise, all technical and scientific terms used herein have the same meaning as commonly understood by one of ordinary skill in the art. Although various methods and materials similar or equivalent to those described herein can be used in the practice or testing of the present invention, suitable methods and materials are described below. However, the skilled artisan understands that the methods and materials used and described are examples and may not be the only ones suitable for use in the invention. Moreover, as measurements are subject to inherent variability, any temperature, weight, volume, time interval, pH, salinity, molarity or molality, range, concentration and any other measurements, quantities or numerical expressions given herein are intended to be approximate and not exact or critical figures unless expressly stated to the contrary.

The term “about,” as used herein, means plus or minus 20 percent of the recited value, so that, for example, “about 0.125” means 0.125±0.025, and “about 1.0” means 1.0±0.2.

The terms “administering” or “administration” of an agent, drug, or peptide to a subject refers to any route of introducing or delivering to a subject a compound to perform its intended function. The administering or administration can be carried out by any suitable route, including orally, intranasally, parenterally (intravenously, intramuscularly, intraperitoneally, or subcutaneously), rectally, or topically. Administering or administration includes self-administration and the administration by another.

“Elevated levels” as used herein in means higher amounts of the nucleic acids or polypeptides of the biomarker that indicates or predicts a need for medical intervention or disease.

“MiR” also “micro RNA” means a class of small non-coding RNAs that are key negative regulators of gene expression Like conventional protein-encoding RNA, miRs are transcribed by RNA polymerase II and their expression is controlled by transcriptional factors. The mature miRs inhibit target mRNA translation or promote their degradation by directly binding to specific miR binding sites in the 3′-untranslated region (3′-UTR) of target genes.

“Biomarker” means a biomolecule (e.g. cytokine, factor, miRNA or other nucleic acids, phospholipid) or blood component that is differentially present (i.e., increased or decreased) in a biological sample from a subject or a group of subjects having a first phenotype (NE) as compared to a biological sample from a subject or group of subjects having a second phenotype (not having NE).

A biomarker may be differentially present at any level, but is generally present at a level that is increased by at least 5%, by at least 10%, by at least 15%, by at least 20%, by at least 25%, by at least 30%, by at least 35%, by at least 40%, by at least 45%, by at least 50%, by at least 55%, by at least 60%, by at least 65%, by at least 70%, by at least 75%, by at least 80%, by at least 85%, by at least 90%, by at least 95%, by at least 100%, by at least 110%, by at least 120%, by at least 130%, by at least 140%, by at least 150%, or more; or is generally present at a level that is decreased by at least 5%, by at least 10%, by at least 15%, by at least 20%, by at least 25%, by at least 30%, by at least 35%, by at least 40%, by at least 45%, by at least 50%, by at least 55%, by at least 60%, by at least 65%, by at least 70%, by at least 75%, by at least 80%, by at least 85%, by at least 90%, by at least 95%, or by 100% (i.e., absent). A biomarker is preferably differentially present at a level that is statistically significant (i.e., a p-value less than 0.05 and/or a q-value of less than 0.10 as determined using either Welch's T-test or Wilcoxon's rank-sum Test).

The term “neonatal encephalopathy (NE)” refers to a syndrome that can occur in newborn babies in which neurological function is disturbed. The most frequent cause of NE is lack of oxygen to the baby at some point during pregnancy or birth. When oxygen deprivation causes NE, the condition may be referred to as “hypoxic ischemic encephalopathy (HIE)”. Lack of oxygen causes damage to the brain, but it can also affect other internal organs. The heart, lungs, kidneys, liver and gastrointestinal system may all experience complications as a result of low oxygen. Babies who experience NE and survive are at high risk to go on to have permanent brain injury or cerebral palsy, but immediate treatment can improve outcomes.

The “level” of one or more biomarkers means the absolute or relative amount or concentration of the biomarker in the sample.

A “reference level” of a biomarker means a level of the biomarker that is indicative of a particular disease state, phenotype, or predisposition to developing a particular disease state or phenotype, or lack thereof, as well as combinations of disease states, phenotypes, or predisposition to developing a particular disease state or phenotype, or lack thereof. A “positive” reference level of a biomarker means a level that is indicative of a particular disease state or phenotype. A “negative” reference level of a biomarker means a level that is indicative of a lack of a particular disease state or phenotype. A “reference level” of a biomarker may be an absolute or relative amount or concentration of the biomarker, a presence or absence of the biomarker, a range of amount or concentration of the biomarker, a minimum and/or maximum amount or concentration of the biomarker, a mean amount or concentration of the biomarker, and/or a median amount or concentration of the biomarker; and, in addition, “reference levels” of combinations of biomarkers may also be ratios of absolute or relative amounts or concentrations of two or more biomarkers with respect to each other. Appropriate positive and negative reference levels of biomarkers for a particular disease state, phenotype, or lack thereof may be determined by measuring levels of desired biomarkers in one or more appropriate subjects, and such reference levels may be tailored to specific populations of subjects (e.g., a reference level may be age-matched or gender-matched so that comparisons may be made between biomarker levels in samples from subjects of a certain age or gender and reference levels for a particular disease state, phenotype, or lack thereof in a certain age or gender group). Such reference levels may also be tailored to specific techniques that are used to measure levels of biomarkers in biological samples where the levels of biomarkers may differ based on the specific technique that is used.

The terms “normal” and “healthy” are used herein interchangeably. They refer to an individual or group of control individuals who have not shown any symptoms of NE damage or diseases. The normal individual (or group of individuals) is not on medication affecting NE damage or diseases. In certain embodiments, normal individuals have similar sex, age, body mass index as compared with the individual from which the sample to be tested was obtained. The term “normal” is also used herein to qualify a sample isolated from a healthy individual.

patient in need

prognosticating

“Sample” or “biological sample” means biological material isolated from a subject. The biological sample may contain any biological material suitable for detecting the desired biomarkers, and may comprise cellular and/or non-cellular material from the subject. The sample can be isolated from any suitable biological fluid such as, for example, blood, blood plasma, blood serum, urine, or cerebral spinal fluid (CSF), tissue or tissue homogenate. Another example of a biological includes EVs obtained from or present in blood serum, or plasma.

The term “sentinel event” as used herein is an unexpected occurrent that results serious physical or psychological injury, or the risk thereof. In a typical example, a sentinel event involves ischemia (such as caused by placental abruption or perinatal asphyxia).

“Severity” of EN refers to the degree of EN on the spectrum of non-EN activity, ranging from mild, moderate, to severe.

The terms “treat”, “treating” or “treatment of” as used herein refers to providing any type of medical management to a subject. Treating includes, but is not limited to, administering a composition to a subject using any known method for purposes such as curing, reversing, alleviating, reducing the severity of, inhibiting the progression of, or reducing the likelihood of a disease, disorder, or condition or one or more symptoms or manifestations of a disease, disorder or condition.

The term “hypothermic treatment” refers to a process used to rapidly lower the body temperature to a near hypothermic state in order to prevent or reduce brain damage. It has many other names, such as “therapeutic hypothermia,” “cooling therapy,” and “neonatal cooling.” Hypothermia therapy involves cooling the baby down to a temperature below homeostasis to allow the brain to recover from a hypoxic-ischemic injury. Typically, the target temperature is about 33.5 degrees Celsius (92.3 degrees Fahrenheit). There are two ways that hypothermia therapy can be administered: using a cooling cap for “selective brain cooling” or by cooling the baby's entire body (“whole-body cooling”).

### 2. Overview

The present invention identifies and analyzes changes in several biomarker associated with NE. Different levels or ratios of these biomarkers (glial fibrillary acidic protein (GFAP), ubiquitin C-terminal hydrolase L1 (UCH-L1), neurofilament light chain (NF-L), and Tau are measured at a single time point or at multiple time points, and used to determine the presence or absence of NE in a patient, the responder or non-responder status of the patient to hypothermia treatment, the severity of the NE, and the prognosis of the patient. MicroRNA biomarkers are also contemplated for use in the invention, and can be used in the same manner as the protein biomarkers. See Table 1 for a list of these miRNA biomarkers. Embodiments of the invention include novel biomarker compounds and compositions, methods of using these biomarkers, and a device for their use.

### 3. Embodiments of the Invention

### A. Biomarkers

A biomarker is defined according to the National Academy of Sciences, as an indicator that signals events in biological samples or systems. Biomarkers are extremely valuable unbiased tools to define the severity and prognosis of NE because they reflect the extent of the nervous system damage and predict neurologic recovery.

1. Protein

The protein biomarkers contemplated for use in the present invention include GFAP, UCH-L1, NF-L, and Tau. Preferably at least two of these markers are used together for testing of a single patient. The preferred protein biomarkers are GFAP and UCH-L1, but the most preferred embodiments of the invention involve testing for all four of these protein biomarkers in the patient.

2. MicroRNA

Based on a 65 miRNA neurology-focused panel, 11 human miRNA were identified in serum which have altered levels in NE (0-6 hours, and/or 48 hours) compared to those in normal control individuals. Importantly, many of these miRNAs are from neurons, glia and astrocytes and are involved in axonal, myelin sheath or synaptic structures in the brain. See Table 1, first 11 biomarkers. Additional biomarkers for NE are also included in the table.

TABLE 1
MicroRNA Biomarkers for NE

| SEQ ID NO | miRNA Name | miRNA Sequence |
|---|---|---|
| 1 | hsa-mir-145-5p | UGAGGUAGUAGUUUGUACAGUU |
| 2 | hsa-mir-16-5p | AGCAGCAUUGUACAGGGCUAUGA |
| 3 | hsa-mir-15a-5p | AGCAGCAUUGUACAGGGCUAUCA |
| 4 | hsa-mir-17-5p | UAACAGUCUACAGCCAUGGUCG |
| 5 | hsa-let-7g-5p | UGUGACUGGUUGACCAGAGGGG |
| 6 | hsa-mir-214-3p | UGUAGUGUUUCCUACUUUAUGGA |
| 7 | hsa-mir-338-3p | GUCCAGUUUUCCCAGGAAUCCCU |
| 8 | hsa-mir-132-3p | UGAGAACUGAAUUCCAUGGGUU |
| 9 | hsa-mir-23a-3p | UCUCCCAACCCUUGUACCAGUG |
| 10 | hsa-mir-26b-5p | CUAGACUGAAGCUCCUUGAGG |
| 11 | hsa-mir-146a-5p | UUAAUGCUAAUCGUGAUAGGGGUU |
| 12 | hsa-mir-150-5p | UAGCAGCACAUAAUGGUUUGUG |
| 13 | hsa-mir-497-5p | UAGCAGCACAUCAUGGUUUACA |
| 14 | hsa-mir-451a | UAGCAGCACGUAAAUAUUGGCG |
| 15 | hsa-mir-29b-3p | CAAAGUGCUUACAGUGCAGGUAG |
| 16 | hsa-mir-323a-3p | CAACGGAAUCCCAAAAGCAGCUG |
| 17 | hsa-mir-107 | UAGCAGCACAGAAAUAUUGGC |
| 18 | hsa-mir-342-3p | UGGAAUGUAAGGAAGUGUGUGG |
| 19 | hsa-mir-382-5p | UAAAGUGCUUAUAGUGCAGGUAG |
| 20 | hsa-mir-486-5p | ACAGCAGGCACAGACAGGCAGU |
| 21 | hsa-mir-103a-3p | AUCACAUUGCCAGGGAUUUCC |
| 22 | hsa-mir-15b-5p | UGGCUCAGUUCAGCAGGAACAG |
| 23 | hsa-mir-20a-5p | UUCAAGUAAUUCAGGAUAGGU |
| 24 | hsa-mir-301a-3p | UAGCACCAUUUGAAAUCAGUGUU |
| 25 | hsa-mir-24-3p | CAGUGCAAUAGUAUUGUCAAAGC |
| 26 | hsa-mir-134-5p | UGUAAACAUCCUUGACUGGAAG |
| 27 | hsa-mir-191-5p | CACAUUACACGGUCGACCUCU |
| 28 | hsa-mir-34a-5p | CUGGCCCUCUCUGCCCUUCCGU |
| 29 | hsa-mir-30e-5p | UCCAGCAUCAGUGAUUUUGUUG |
| 30 | hsa-mir-93-5p | UCUCACACAGAAAUCGCACCCGU |
| 31 | hsa-mir-206 | UGGCAGUGUCUUAGCUGGUUGU |
| 32 | hsa-mir-151a-3p | GAAGUUGUUCGUGGUGGAUUCG |
| 33 | hsa-mir-195-5p | AAACCGUUACCAUUACUGAGUU |
| 34 | hsa-mir-155-5p | UCCUGUACUGAGCUGCCCCGAG |
| 35 | hsa-mir-328-3p | CAGCAGCACACUGUGGUUUGU |
| 36 | hsa-mir-885-5p | UGGAAGACUAGUGAUUUUGUUGUU |
| 37 | hsa-mir-7-5p | UCCAUUACACUACCCUGCCUCU |
| 38 | hsa-mir-142-3p | CAAAGUGCUGUUCGUGCAGGUAG |
| 39 | hsa-mir-98-5p | UGAGGUAGUAAGUUGUAUUGUU |

3. Panels of Biomarkers

In preferred embodiments of the invention, a panel of at least the four protein NE biomarkers is used, including, GFAP. UCH-L1, NF-1, and Tau. In additional embodiments, GFAP and UCH-L1 are used as the biomarkers, optionally with other biomarkers such as NF-L, Tau, hsa-mir-145-5p, hsa-mir-16-5p, hsa-mir-15a-5p, hsa-mir-17-5p, hsa-let-7g-5p, hsa-mir-214-3p, hsa-mir-338-3p, hsa-mir-132-3p, hsa-mir-23a-3p, hsa-mir-26b-5p and hsa-mir-146a-5p, hsa-mir-150-5p, hsa-mir-497-5p, hsa-mir-451a, hsa-mir-29b-3p, hsa-mir-323a-3p, hsa-mir-107, hsa-mir-342-3p, hsa-mir-382-5p, hsa-mir-486-5p, hsa-mir-103a-3p, hsa-mir-15b-5p, hsa-mir-20a-5p, hsa-mir-301a-3p, hsa-mir-301a-3p, hsa-mir-24-3p, hsa-mir-134-5p, hsa-mir-191-5p, hsa-mir-34a-5p, hsa-mir-30e-5p, hsa-mir-93-5p, hsa-mir-206, hsa-mir-151a-3p, hsa-mir-195-5p, hsa-mir-155-5p, hsa-mir-328-3p, hsa-mir-885-5p, hsa-mir-7-5p, hsa-mir-142-3p, hsa-mir-98-5p.

A panel of the biomarkers can be used together to aid in identifying particular injured brain regions, the severity of injury, and aid in identifying components of the pathophysiologic cascade. Using the biomarkers in a panel or in combination provides a more powerful NE diagnostic and prognostic tool.

The invention employs biomarkers indicative of NE to develop a method for a personalized medical approach to diagnosis and treatment of NE in neonates and to monitor neonates' responses to therapeutic hypothermia. Using a biomarker panel in neonates with NE according to embodiments of the invention aid in neonatal direct care by providing a rapid test to predict outcomes, to select candidates who are likely to benefit from therapeutic hypothermia, and to gauge the response to this neuroprotective intervention.

In addition, biomarker profiles according to the invention allow monitoring of the individualized application of other neuroprotective agents or NE treatments. For example, as new therapies such as Xenon, currently in early clinical trials, are being developed, the biomarker profile studies outlined here can allow clinicians, for the first time, to determine those patients unlikely to benefit from hypothermia alone and become the ideal subject for new or adjunctive therapies. The power of clinical trials will be enhanced by the ability of clinicians to study a more homogeneous population, excluding patients who are unlikely to benefit from the new or adjunct therapy. This individualized approach represents an improvement from the current “one size fits all” treatment for neonates with NE. In addition, embodiments of the invention also include a point of care analytical device to measure a combination of biomarkers in the same sample, allowing clinicians to obtain biomarkers testing and rapid results to the bedside.

### B. Biological Samples

1. Blood, Serum, Plasma, CSF and the Like

The biomarker levels can be measured in one or more biofluid samples taken from the patient, including blood, blood plasma, blood serum, cerebrospinal fluid (CSF), and the like, or from solid biosamples selected from biopsy or autopsy nervous system tissue samples. Samples of blood, serum, plasma, or CSF can be used as is or diluted. If needed, the biological samples can be partially purified before analysis, for example by chromatography or the like.

At least one sample is taken between 0 and 6 hours after birth. In preferred embodiments, more than one sample is taken for testing. For example, 2, 3, 4, 5, 6, 7, or 8 samples can be taken at different times after birth, generally between 0 and 96 hours after birth. Suitable sampling intervals are 0-6 hours after birth, 24 hour after birth, 48 hours after birth, and 72 hours after birth, however the skilled practitioner can determine convenient intervals for sampling based on the individual condition of the patient involved.

Normal plasma levels in neonates for the protein biomarkers of this invention are GFAP (352.8±53 pg/ml), UCHL1 (413±32.83 pg/ml), NFL (11.72±1 pg/ml), Tau (17.33±3.28 pg/ml), respectively. The results of testing for the biomarkers are compared to the normal (control) levels. A GFAP plasma level of at least 425, 500, or 600 pg/ml (e.g., 1081±132.4, 1815±625.9, 1345±436.8, 2312±112, 2173±1159, measurements taken at 0-6h, 12h, 16h, 24h and 96h, respectively), a UCHL1 plasma level of at least 460, 500, or 600 pg/ml (e.g., 1342±538.7, 667.5±160.4, 352.7±56.76, 272.4±60.3, 372±146.1, measurements taken at 0-6h, 12h, 16h, 24h and 96h, respectively), NFL plasma level of least 15, 50, 100 pg/ml (e.g. 115.2±38.61, 116.8±28.89, 135.6±32.58, 1487±1120, 446.3±89.43, measurements taken at 0-6h, 12h, 16h, 24h and 96h, respectively), and Tau plasma level of at least 25, 35, or 50 pg/ml (e.g. 64.31±20.04, 23.1±5.96, 21.75±6.82, 30.88±9.09, 70.45±29.50, measurements taken at 0-6h, 12h, 16h, 24h and 96h, respectively) are considered elevated over these controls.

Normal levels for the miRNA biomarkers in neonates are hsa-let-7g-5p (1505.942±475), hsa-mir-103a-3p (2864.07±868.27), hsa-mir-107 (2309.534±590.541), hsa-mir-132-3p (11.012±40.924), hsa-mir-134-5p (76.966±61.986), hsa-mir-142-3p (32.402±25.233), hsa-mir-145-5p (248.208±421.205), hsa-mir-146a-5p (4933.712±2554.302), hsa-mir-150-5p (489.051±520.563), hsa-mir-151a-3p (288.217±95.972), hsa-mir-155-5p (15.876±14.556), hsa-mir-15a-5p (7466.099±1822.202), hsa-mir-15b-3p (90.809±57.011), hsa-mir-16-2-3p (19.167±17.371), hsa-mir-17-5p (25260.177±5288.907), hsa-mir-191-5p (2256.432±794.261), hsa-mir-195-5p (5610.915±1844.393), hsa-mir-206 (0.256±18.161), hsa-mir-20a-5p (12980.853±2841.911), hsa-mir-214-3p (200.793±248.09), hsa-mir-23a-3p (2088.084±1161.918), hsa-mir-24-3p (2239.294±1494.405), hsa-mir-26b-5p (91.042±67.003), hsa-mir-29b-3p (245.758±144.565), hsa-mir-301a-3p (107.566±80.468), hsa-mir-30e-5p (14.69±6.888), hsa-mir-323a-3p (204.979±167.429), hsa-mir-328-3p (271.59±138.841), hsa-mir-338-3p (26.995±33.658), hsa-mir-342-3p (405.741±400.772), hsa-mir-34a-5p (76.042±135.389), hsa-mir-382-5p (1230.575±491.271), hsa-mir-451a (78460.02±24405.544), hsa-mir-486-5p (25782.134±7250.617), hsa-mir-497-5p (1363.343±409.608), hsa-mir-7-5p (258.837±154.62), hsa-mir-885-5p (6.361±2402.438 or alternatively 39.84±33.85), hsa-mir-93-5p (22894.369±5963.523), hsa-mir-98-5p (167.63±58.632), respectively.

Normal levels for the miRNA biomarkers of the invention are hsa-let-7g-5p(1505.942±475), hsa-mir-103a-3p(2864.07±868.27), hsa-mir-107(2309.534±590.541), hsa-mir-132-3p(11.012±40.924), hsa-mir-134-5p(76.966±61.986), hsa-mir-142-3p(32.402±25.233), hsa-mir-145-5p(248.208±421.205), hsa-mir-146a-5p(4933.712±2554.302), hsa-mir-150-5p(489.051±520.563), hsa-mir-151a-3p(288.217±95.972), hsa-mir-155-5p(15.876±14.556), hsa-mir-15a-5p(7466.099±1822.202), hsa-mir-15b-3p(90.809±57.011), hsa-mir-16-2-3p(19.167±17.371), hsa-mir-17-5p(25260.177±5288.907), hsa-mir-191-5p(2256.432±794.261), hsa-mir-195-5p(5610.915±1844.393), hsa-mir-206(0.256±18.161), hsa-mir-20a-5p(12980.853±2841.911), hsa-mir-214-3p(200.793±248.09), hsa-mir-23a-3p(2088.084±1161.918), hsa-mir-24-3p(2239.294±1494.405), hsa-mir-26b-5p(91.042±67.003), hsa-mir-29b-3p(245.758±144.565), hsa-mir-301a-3p(107.566±80.468), hsa-mir-30e-5p(14.69±6.888), hsa-mir-323a-3p(204.979±167.429), hsa-mir-328-3p(271.59±138.841), hsa-mir-338-3p(26.995±33.658), hsa-mir-342-3p(405.741±400.772), hsa-mir-34a-5p(76.042±135.389), hsa-mir-382-5p(1230.575±491.271), hsa-mir-451a(78460.02±24405.544), hsa-mir-486-5p(25782.134±7250.617), hsa-mir-497-5p(1363.343±409.608), hsa-mir-7-5p(258.837±154.62), hsa-mir-885-5p(6.361±2402.438, or alternatively 39.84±33.85), hsa-mir-93-5p(22894.369±5963.523), hsa-mir-98-5p(167.63±58.632), respectively.

Using the same order of the miRNAs provided above, a result indicating a deviation from the normal level is, for example, 1505.942±475±831.883, 2864.07±868.27±1807.587, 2309.534±590.541±1241.855, 11.012±40.924±96.643, 76.966±61.986±288.794, 32.402±25.233±88.82, 248.208±421.205±612.689, 4933.712±2554.302±2501.781, 489.051±520.563±802.452, 288.217±95.972±350.856, 15.876±14.556±26.075, 7466.099±1822.202±1681.273, 90.809±57.011±44.365, 19.167±17.371±7.069, 25260.177±5288.907±4135.132, 2256.432±794.261±1894.076, 5610.915±1844.393±1937.084, 0.256±18.161±1253.213, 12980.853±2841.911±2238.159, 200.793±248.09±593.96, 2088.084±1161.918±1412.044, 2239.294±1494.405±1395.498, 91.042±67.003±84.512 245.758±144.565±161.51, 107.566±80.468±85.404, 14.69±6.888±19.8, 204.979±167.429±920.458, 271.59±138.841±417.802, 26.995±33.658±38.261, 405.741±400.772±374.277, 76.042±135.389±480.606, 1230.575±491.271±2226.13, 78460.02±24405.544±15143.368, 25782.134±7250.617±7604.478, 1363.343±409.608±1542.924, 258.837±154.62±145.391, 6.361±2402.438±820.711, 22894.369±5963.523±4053.536, 167.63±58.632±54.837 at 6h in order to be considered elevated or declined over the controls.

Using the same order of miRNAs above, 1567.654±730.512, 4116.668±1504.576, 3003.317±876.577, 122.457±63.942, 69.283±105.876, 52.23±40.247, 143.696±251.269, 5421.923±2250.934, 707.461±415.114, 700.678±219.251, 7.229±21.825, 7667.647±2459.853, 102.305±78.685, v3.338±14.206, 17069.811±6524.642, 4282.783±1411.306, 4356.186±3675.792, 12.627±611.72, 10600.994±3641.483, 87.524±355.243, 2753.252±1081.65, 2551.02±1313.556, 132.677±88.499, 295.356±128.215, 146.738±92.936, 7.792±9.779, 623.418±789.774, 399.884±282.979, 12.939±29.794, 422.543±280.121, 23.999±119.51, 1219.556±1287.816, 28027.195±11405.861, 13093.183±5892.258, 1300.011±851.858, 329.193±304.608, 5.73±240.171, 17112.423±5983.965, 54.004±51.067 at 48h in order to be considered elevated or declined over these controls.

The methods for detection of the biomarkers include any testing method known in the art and which is convenient for the user. Typically, antibody methods are used for specific detection and quantitation of proteins such as the biomarkers here. Assays therefore include ELISA assays, RIA assays, sandwich assays, and the like, using a specific antibody or aptamer to specifically detect individual biomarkers. Other suitable assay methods include electrochemical and fluorescence-based detection, or immuno-amplification assays (e.g. immuno-Polymerase Chain Reaction (PCR) or rolling cycle amplification (RCA)). Methods for detection of miRNA are known in the art and include qPCR, miRNA arrays, RNA-seq, multiplex miRNA profiling, and the like. In a specific embodiment, the method(s) for detection and quantitation of miRNA biomarkers are, Point-of Care device-based method, Core Medical Lab PCR-based detection or bead-based flow cytometer-based sorting/amplification method.

### C. Methods of Use

The proposed combinatory protein and/or miRNA biomarker panel measurable in biological samples can be used for the following clinically relevant purposes:

1. Diagnosis: The inventive combinatory protein and/or miRNA biomarker panel can be used to aid in the diagnosis of NE in addition to the following tools
1) Low APGAR Scores: An APGAR score of less than 5 at 5 minutes and 10 minutes may indicate encephalopathy.
2) An abnormal neurologic exam include changes in mental status (decreased alertness); Increased or decreased muscle tone; Seizures; Abnormal pupils; Changes in reflexes; Changes in breathing and heart rate; Cord blood samples (cord blood can show the umbilical arterial and venous pH. Acute brain injury seen on an MRI that shows hypoxic-ischemia); EEG (this test records the activity of the baby's brain by measuring electrical currents through the brain. This can distinguish seizures from other issues and determine their cause). Ultrasound (this test uses sound waves to evaluate echo in the brain suggestive of cerebral edema and ischemia. This can suggest the timing and extent of NE and can locate hemorrhages and determine ventricular size with acute sensitivity).

2. Severity assessment: The inventive combinatory protein and/or miRNA biomarker panel can be used to aid in the assessment of NE severity in addition or coupled to Sarnat stage I (mild NE), II (moderate NE), III (severe NE).

3. Prognosis: The inventive combinatory protein and/or miRNA biomarker panel can be used to aid in the assessment of NE patient outcome. A protein and/or miRNA biomarker panel can be combined with the HIE Bayler outcome scores including cognitive function, language function and motor function category scores. Bayler category scores that are less than 85 indicate a poor outcome. Bayler category scores equal to or greater than 85 indicate a good outcome.

4. Treatment decision and response prediction: The inventive combinatory protein and/or miRNA biomarker panel can be used to aid in the NE treatment decision. For example, NE patients are candidates for hypothermia/brain cooling therapy. The use of the proposed combinatory protein and/or miRNA biomarker panel in a point of care setting can help the physicians make clinical decisions as to whether hypothermia/brain cooling therapy should be administered. In addition, the proposed combinatory protein and/or miRNA biomarker panel when measured repeatedly can be used to track patient response to NE treatment/therapy.

### D. Devices/Kits and Testing Methods

A preferred embodiment of the invention is a diagnostic kit or point-of-care (POC) testing method which allows the clinician to test for one or more of the biomarkers discussed herein to obtain rapid information about the levels of differentially expressed proteins in the samples of patients with NE. The testing can be repeated at different time points and with different biomarkers or panels of biomarkers to obtain further information. The results of the testing can be compared to normal control levels and to the results from previous tests on the same patient. The device or kit also includes instructions for use and optionally written material containing normal or control levels of specific biomarkers for patients with different degrees of NE at different times after the precipitating event or birth. A determination of the severity and/or prognosis of NE is made by comparing levels of the biomarkers in the patient samples and control or exemplary ranges in the instructions said proteins in an injured patient to the protein levels in the tables.

1. In Vitro Diagnostic Device

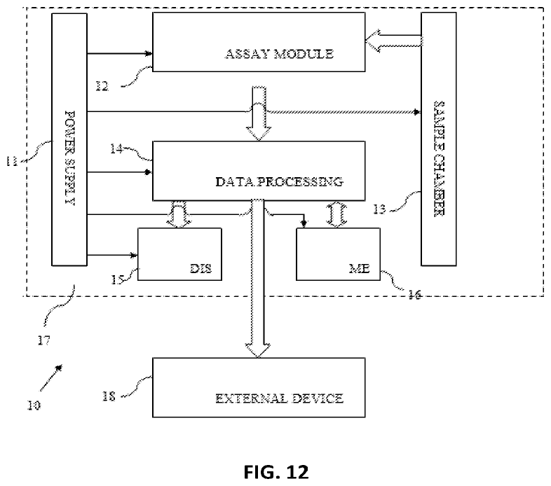

FIG. 12 schematically illustrates an inventive in vitro diagnostic device shown generally at 10. An inventive in vitro diagnostic device includes at least a sample collection chamber 13, an assay module 12 used to detect biomarkers of injury, disease or repair, and a user interface that relates the concentration (level) of the measured biomarker measured in the assay module. The in vitro diagnostic device may be a handheld device, a bench top device, or a point of care device.

The sample chamber 13 can be of any sample collection apparatus known in the art for holding a biological fluid. In one embodiment, the sample collection chamber can accommodate any one of the biological fluids herein contemplated, such as whole blood, plasma, serum, urine, sweat or saliva.

The assay module 12 is preferably made of an assay which may be used for detecting a protein antigen in a biological sample, for instance, through the use of antibodies in an immunoassay. The assay module 12 may include any assay currently known in the art; however, the assay should be optimized for the detection of NE biomarkers used for diagnosing NE, severity of injury, or responsiveness to therapy in a subject. The assay module 12 is in fluid communication with the sample collection chamber 13. In one embodiment, the assay module 12 is configured to conduct an immunoassay where the immunoassay may be any one of a radioimmunoassay, ELISA (enzyme linked immunosorbent assay), “sandwich” immunoassay, immunoprecipitation assay, precipitin reactions, gel diffusion precipitin reactions, immunodiffusion assay, fluorescent immunoassay, chemiluminescent immunoassay, phosphorescent immunoassay, or an anodic stripping voltammetry immunoassay. Alternatively, the assay module is configured to conduct a nucleic acid hybridization assay. In one embodiment a colorimetric assay may be used which may include only of a sample collection chamber 13 and an assay module 12 of the assay. Although not specifically shown these components are preferably housed in one assembly 17.

In one embodiment, the inventive in vitro diagnostic device contains a power supply 11, an assay module 12, a sample chamber 13, and a data processing module 14. The power supply 11 is electrically connected to the assay module and the data processing module 14. The assay module 12 and the data processing module 14 are in electrical communication with each other. As described above, the assay module 12 may include any assay currently known in the art; however, the assay should be optimized for the detection of the biomarkers used herein for detecting injury disease, or repair in a subject. The assay module 12 is in fluid communication with the sample collection chamber 13. The assay module 12 includes of an immunoassay where the immunoassay may be any one of a radioimmunoassay, ELISA (enzyme linked immunosorbent assay), “sandwich” immunoassay, immunoprecipitation assay, precipitin reactions, gel diffusion precipitin reactions, immunodiffusion assay, fluorescent immunoassay, chemiluminescent immunoassay, phosphorescent immunoassay, or an anodic stripping voltammetry immunoassay. A biological sample is placed in the sample chamber 13 and assayed by the assay module 12 detecting for a biomarker of injury, disease, or repair. The measured amount of the biomarker by the assay module 12 is then electrically communicated to the data processing module 14. The data processing 14 module may include any known data processing element known in the art, and may include a chip, a central processing unit (CPU), or a software package which processes the information supplied from the assay module 12.

In one embodiment, the data processing module 14 is in electrical communication with a display 15, a memory device 16, or an external device 18 or software package [such as laboratory and information management software (LIMS)]. In one embodiment, the data processing module 14 is used to process the data into a user defined usable format. This format includes the measured concentration (levels) of NE biomarkers detected in the sample, and that are useful for diagnosing NE, severity of injury, or responsiveness to therapy in a subject. The information from the data processing module 14 may be illustrated on the display 15, saved in machine readable format to a memory device, or electrically communicated to an external device 18 for additional processing or display. Although not specifically shown these components are preferably housed in one assembly 17. In one embodiment, the data processing module 14 may be programmed to compare the detected amount of the biomarker transmitted from the assay module 12, to a comparator algorithm. The comparator algorithm may compare the measured amount to the user defined threshold which may be any limit useful by the user. In one embodiment, the user defined threshold is set to the amount of the biomarker measured in control subject, or a statistically significant average of a control population.

In one embodiment, an in vitro diagnostic device may include one or more devices, tools, and equipment configured to hold or collect a biological sample from an individual. In one embodiment of an in vitro diagnostic device, tools to collect a biological sample may include one or more of a swab, a scalpel, a syringe, a scraper, a container, and other devices and reagents designed to facilitate the collection, storage, and transport of a biological sample. In one embodiment, an in vitro diagnostic test may include reagents or solutions for collecting, stabilizing, storing, and processing a biological sample. These reagents include antibodies, aptamers, or combinations thereof raised against one of the aforementioned biomarkers. In one embodiment, an in vitro diagnostic device, as disclosed herein, may include a micro array apparatus and reagents, and additional hardware and software necessary to assay a sample to detect and visualize the temporally relevant biomarkers.

2. Kits

In yet another aspect, disclosed are kits for aiding a diagnosis of injury, disease, or repair, including type, phase amplitude (severity), subcellular localization, wherein the kits may be used to detect the markers of the present invention. For example, the kits can be used to detect any one or more of the biomarkers described herein, which markers are differentially present in samples of a patient and normal subjects. In another example, the kits can be used to identify compounds that modulate expression of one or more of the markers in in vitro or in vivo animal models to determine the effects of treatment.

In one embodiment, a kit includes (a) an antibody, aptamer, or nucleic acid probe that specifically binds to an aforementioned marker; and (b) a detection reagent. Such kits are prepared from the materials described above, and the previous discussion regarding the materials (e.g., antibodies, aptamers detection reagents, immobilized supports, etc.) being fully applicable to this section and thus is not repeated.

In one inventive embodiment, the kit includes (a) a panel or composition of detecting agent to detect a panel or composition of biomarkers. The panel or composition of reagents included in a kit provide for the ability to detect at least one each of the early, intermediate, and late biomarkers in order to diagnose an injury, disease or repair event. These biomarkers corresponding to at least one each of early, intermediate, and late phases of the injury, disease or repair process as detailed in Table 1 as shown below in example 3.

In one embodiment, the invention includes a diagnostic kit for use in screening serum containing antigens of the biomarkers of the invention. The diagnostic kit in this embodiment includes a substantially isolated antibody or aptamer specifically immunoreactive with peptide or polynucleotide antigens, or nucleic acid probes that hybridize with polynucleotide biomarkers, and visually detectable labels associated with the binding of the polynucleotide or peptide antigen to the antibody or aptamer or nucleic acid probe. In one embodiment, the antibody or aptamer is attached to a solid support. Antibodies or aptamers used in the inventive kit are those raised against any one of the biomarkers used herein for temporal data. In one embodiment, the antibody is a monoclonal or polyclonal antibody or aptamer raised against the rat, rabbit or human forms of the biomarker. The detection reagent of the kit includes a second, labeled monoclonal or polyclonal antibody or aptamer. Alternatively, or in addition thereto, the detection reagent includes a labeled, competing antigen.

In one diagnostic configuration, test serum is reacted with a solid phase reagent having a surface-bound antigen obtained by the methods of the present invention. After binding with specific antigen antibody or aptamer to the reagent and removing unbound serum components by washing, the reagent is reacted with reporter-labeled anti-human antibody or aptamer to bind reporter to the reagent in proportion to the amount of bound anti-antigen antibody or aptamer on the solid support. The reagent is again washed to remove unbound labeled antibody or aptamer, and the amount of reporter associated with the reagent is determined. Typically, the reporter is an enzyme which is detected by incubating the solid phase in the presence of a suitable fluorometric, luminescent or colorimetric substrate.

The solid surface reagent in the above assay is prepared by known techniques for attaching protein or oligonucleotide material to solid support material, such as polymeric beads, dip sticks, 96-well plate or filter material. These attachment methods generally include non-specific adsorption of the protein oligonucleotide to the support or covalent attachment of the protein or oligonucleotide, typically through a free amine group, to a chemically reactive group on the solid support, such as an activated carboxyl, hydroxyl, or aldehyde group. Alternatively, streptavidin coated plates can be used in conjunction with biotinylated antigen(s).

In some embodiments, the kit may include a standard or control information so that the test sample can be compared with the control information standard to determine if the test amount of a marker detected in a sample is a diagnostic amount consistent with a diagnosis of injury, disease, or repair, including type, phase, amplitude (severity), subcellular localization, brain disorder and/or effect of treatment on the patient.

In one embodiment, a kit includes: (a) a substrate including an adsorbent thereon, wherein the adsorbent is suitable for binding a marker, and (b) instructions to detect the marker or markers by contacting a sample with the adsorbent and detecting the marker or markers retained by the adsorbent. In some embodiments, the kit may include an eluant (as an alternative or in combination with instructions) or instructions for making an eluant, wherein the combination of the adsorbent and the eluant allows detection of the markers using gas phase ion spectrometry. Such kits can be prepared from the materials described above, and the previous discussion of these materials (e.g., probe substrates, adsorbents, washing solutions, etc.) is fully applicable to this section and will not be repeated.

In certain embodiments, the kit further includes instructions for suitable operational parameters in the form of a label or a separate insert. For example, the kit may have standard instructions informing a consumer how to wash the probe after a sample is contacted on the probe. In another example, the kit may have instructions for pre-fractionating a sample to reduce complexity of proteins in the sample. In another example, the kit may have instructions for automating the fractionation or other processes.

3. Biofluids

The inventive method and in vitro diagnostic devices provide the ability to detect and monitor levels of those of NE biomarkers which are released into the body after ischemic event provide enhanced diagnostic capability by allowing clinicians (1) to determine the type, phase and amplitude (severity) of injury or disease or repair in various patients, (2) to monitor patients for signs of NE symptoms and (3) to continually monitor the progress of the injury, disease, or repair and the effects of therapy by examination of these NE biomarkers in biological fluids (synonymously referred to herein as “biofluids”), such as blood, plasma, serum, CSF, urine, saliva or sweat. Unlike other organ-based diseases where rapid diagnostics by surrogate biomarkers prove invaluable to the course of action taken to treat the disease, no such rapid, definitive diagnostic tests exist for ischemic event that might provide physicians with quantifiable NE biomarkers to help determine the degree of the injury, disease or repair; the anatomical and cellular pathology of the injury, disease or repair; and the implementation of appropriate medical management and treatment.

A biological sample operative herein includes cells, tissues, cerebral spinal fluid (CSF), whole blood, serum, plasma, cytosolic fluid, urine, feces, stomach fluids, digestive fluids, saliva, nasal or other airway fluid, vaginal fluids, semen, or other biological fluid recognized in the art. It should be appreciated that after an ischemic event, the neural cell membrane is compromised, leading to the efflux of neural proteins first into the extracellular fluid, and to the cerebrospinal fluid. Eventually the neural proteins efflux to the circulating blood (as assisted by the compromised blood brain barrier for brain injuries or diseases) and, through normal bodily function (such as impurity removal from the kidneys), the neural proteins migrate to other biological fluids such as urine, sweat, and saliva. Thus, other suitable biological samples include, but are not limited to such cells or fluid secreted from these cells. It should also be appreciated that obtaining biological fluids such as cerebrospinal fluid, blood, plasma, serum, saliva, and urine, from a subject is typically much less invasive and traumatizing than obtaining a solid tissue biopsy sample. Thus, biofluids, are preferred for use in the invention.

Biological samples of CSF, blood, urine, and saliva are collected using normal collection techniques. For example, and not to limit the sample collection to the procedures contained herein, CSF Lumbar Puncture (LP) a 20-gauge introducer needle is inserted, and an amount of CSF is withdrawn. For blood, the samples may be collected by venipuncture in Vacutainer tubes and being amenable to being spun down and separated into serum and plasma. For urine and saliva, samples that are collected avoiding the introduction of contaminants into the specimen are preferred. All biological samples may be stored in aliquots at −80° C. for later assay. Surgical techniques for obtaining solid tissue samples are well known in the art. For example, methods for obtaining a nervous system tissue sample are described in standard neuro-surgery texts such as Atlas of Neurosurgery: Basic Approaches to Cranial and Vascular Procedures, by F. Meyer, Churchill Livingstone, 1999; Stereotactic and Image Directed Surgery of Brain Tumors, 1st ed., by David G. T. Thomas, WB Saunders Co., 1993; and Cranial Microsurgery: Approaches and Techniques, by L. N. Sekhar and E. De Oliveira, 1st ed., Thieme Medical Publishing, 1999. Methods for obtaining and analyzing brain tissue are also described in Belay et al., Arch. Neurol. 58: 1673-1678 (2001); and Seijo et al., J. Clin. Microbiol. 38: 3892-3895 (2000). Any suitable biological samples can be obtained from a subject to detect markers. It should be appreciated that the methods employed herein may be identically reproduced for any biological fluid to detect a marker or markers in a sample.

After an ischemic event, the damaged tissue, organs, or nerve cells in in vitro culture or in situ in a subject express altered levels or activities of one or more proteins than do such cells not subjected to the insult. Thus, samples that contain nerve cells, e.g., a biopsy of CNS or PNS tissue are illustratively suitable biological samples for use in the invention.

A subject illustratively includes a dog, a cat, a horse, a cow, a pig, a sheep, a goat, a chicken, non-human primate, a human, a rat, and a mouse. Subjects who most benefit from the present invention are neonates who are suspected of having experienced an ischemic event such as those aforementioned herein.

Baseline levels of several biomarkers are those levels obtained in the target biological sample in the species of desired subject in the absence of a known injury, disease, or repair. These levels need not be expressed in hard concentrations but may instead be known from parallel control experiments and expressed in terms of fluorescent units, density units, and the like. Typically, baselines are determined from subjects where there is an absence of a biomarker or present in biological samples at a negligible amount. However, some proteins may be expressed less in an injured, diseased or repaired patient or before any clinical measures of injury, disease, or repair. Determining the baseline levels of protein biomarkers in a particular species is well within the skill of the art.

To provide correlations between an injury, disease, or repair and measured quantities of the NE biomarkers, biological samples are collected from subjects in need of measurement for these biomarkers to assess injury, disease, or repair. Detected levels of a given NE biomarker are optionally correlated with CT scan results as well as GCS scoring.

The detection methods may be implemented into assays or into kits for performing assays. These kits or assays may alternatively be packaged into a cartridge to be used with an inventive in vitro diagnostic device. Such a device makes use of these cartridges, kits, or assay in an assay module 12, which may be one of many types of assays. The biomarkers of the invention can be detected in a sample by a variety of conventional methods. For example, immunoassays, include but are not limited to competitive and non-competitive assay systems using techniques such as western blots, radioimmunoassay, ELISA (enzyme linked immunosorbent assay), “sandwich” immunoassays, magnetic immunoassays, radioisotope immunoassay, fluorescent immunoassays, immunoprecipitation assays, precipitin reactions, gel diffusion precipitin reactions, immunodiffusion assays, fluorescent immunoassays, chemiluminescent immunoassays, phosphorescent immunoassays, anodic stripping voltammetry immunoassay, and the like. Inventive in vitro diagnostic devices may also include any known devices currently available that utilize ion-selective electrode potentiometry, microfluids technology, fluorescence or chemiluminescence, or reflection technology that optically interprets color changes on a protein test strip. Such assays are routine and well known in the art (see, e.g., Ausubel et al., eds, 1994, Current Protocols in Molecular Biology, Vol. 1, John Wiley & Sons, Inc., New York, which is incorporated by reference herein in its entirety). Exemplary immunoassays are described briefly below (but are not intended by way of limitation). It should be appreciated, that at present, none of the existing technologies present a method of detecting or measuring any of the ailments disclosed herein, nor does there exist any methods of using such in vitro diagnostic devices to detect any of the disclosed biomarkers to detect their associated injuries.

An exemplary process for detecting the presence or absence of a biomarker, alone or in combination, in a biological sample involves obtaining a biological sample from a subject, such as a human, contacting the biological sample with a compound or an agent capable of detecting of the marker being analyzed, illustratively including an antibody or aptamer, and analyzing binding of the compound or agent to the sample after washing. Those samples having specifically bound compound or agent express the marker being analyzed.

For example, in vitro techniques for detection of a marker illustratively include enzyme linked immunosorbent assays (ELISAs), radioimmunoassay, radioassay, western blot, Southern blot, northern blot, immunoprecipitation, immunofluorescence, mass spectrometry, RT-PCR, PCR, liquid chromatography, high performance liquid chromatography, enzyme activity assay, cellular assay, positron emission tomography, mass spectroscopy, combinations thereof, or other technique known in the art. Furthermore, in vivo techniques for detection of a marker include introducing a labeled agent that specifically binds the marker into a biological sample or test subject. For example, the agent can be labeled with a radioactive marker whose presence and location in a biological sample or test subject can be detected by standard imaging techniques. In some inventive embodiments a first NE biomarker early, intermediate, and late specific binding agent and other agents specifically binding at least one additional NE biomarker are bound to a substrate. It is appreciated that a bound agent assay is readily formed with the agents bound with spatial overlap, with detection occurring through discernibly different detection of each NE biomarkers. A color intensity-based quantification of each of the spatially overlapping bound biomarkers is representative of such techniques.

A preferred agent for detecting a NE biomarker is an antibody, aptamer or nucleic acid probe sequence capable of binding to the biomarker being analyzed. More preferably, the antibody, aptamer or nucleic acid probe sequence is conjugated with a detectable label. Such antibodies can be polyclonal or monoclonal. An intact antibody, a fragment thereof (e.g., Fab or F(ab′)₂), or an engineered variant thereof (e.g., sFv) or an aptamer or bi-/tri-specific aptamer can also be used. Such antibodies can be of any immunoglobulin class including IgG, IgM, IgE, IgA, IgD and any subclass thereof. Antibodies and aptamers for numerous inventive biomarkers are available from vendors known to one of skill in the art. Exemplary antibodies operative herein are used to detect a biomarker of the disclosed conditions. In addition, antigens to detect autoantibodies may also be used to detect late injury of the stated injuries and disorders.

An antibody or aptamer is labeled in some inventive embodiments. A person of ordinary skill in the art recognizes numerous labels operable herein. Labels illustratively include, fluorescent labels, biotin, peroxidase, radionucleotides, or other label known in the art. Alternatively, a detection species of another antibody or aptamer or other compound known to the art is used as form detection of a biomarker bound by an antibody or aptamer.

Antibody- and aptamer-based assays operative herein include western blotting immunosorbent assays (e.g., ELISA and RIA) and immunoprecipitation assays. As one example, the biological sample or a portion thereof is immobilized on a substrate, such as a membrane made of nitrocellulose or PVDF; or a rigid substrate made of polystyrene or other plastic polymer such as a microtiter plate, and the substrate is contacted with an antibody or aptamer that specifically binds a NE biomarker under conditions that allow binding of antibody or aptamer to the biomarker being analyzed. After washing, the presence of the antibody or aptamer on the substrate indicates that the sample contained the marker being assessed. If the antibody or aptamer is directly conjugated with a detectable label, such as an enzyme, fluorophore, or radioisotope, the presence of the label is optionally detected by examining the substrate for the detectable label. Alternatively, a detectably labeled secondary antibody or aptamer that binds the marker-specific antibody or aptamer is added to the substrate. The presence of detectable label on the substrate after washing indicates that the sample contained the biomarker.

Numerous permutations of these basic immunoassays are also operative in the invention. These include the biomarker-specific antibody or aptamer, as opposed to the sample being immobilized on a substrate, and the substrate is contacted with a biomarker conjugated with a detectable label under conditions that cause binding of antibody or aptamer to the labeled marker. The substrate is then contacted with a sample under conditions that allow binding of the marker being analyzed to the antibody or aptamer. A reduction in the amount of detectable label on the substrate after washing indicates that the sample contained the marker.

Although antibodies or aptamers are preferred for use in the invention because of their extensive characterization, any other suitable agent (e.g., a peptide or a small organic molecule) that specifically binds a biomarker is operative herein in place of the antibody or aptamer in the above described immunoassays. Methods for making aptamers with a particular binding specificity are known as detailed in U.S. Pat. Nos. 5,475,096; 5,670,637; 5,696,249; 5,270,163; 5,707,796; 5,595,877; 5,660,985; 5,567,588; 5,683,867; 5,637,459; and 6,011,020.

A myriad of detectable labels that are operative in a diagnostic assay for biomarker expression are known in the art. Agents used in methods for detecting a biomarker are conjugated to a detectable label, e.g., an enzyme such as horseradish peroxidase. Agents labeled with horseradish peroxidase may be detected by adding an appropriate substrate that produces a color change in the presence of horseradish peroxidase. Several other detectable labels that may be used are known. Common examples of these detectable labels include alkaline phosphatase, horseradish peroxidase, fluorescent compounds, luminescent compounds, colloidal gold, magnetic particles, biotin, radioisotopes, and other enzymes. It is appreciated that a primary/secondary antibody or aptamer system is optionally used to detect one or more biomarkers. A primary antibody or aptamer that specifically recognizes one or more biomarkers is exposed to a biological sample that may contain the biomarker of interest. A secondary antibody or aptamer with an appropriate label that recognizes the species or isotype of the primary antibody or aptamer is then contacted with the sample such that specific detection of the one or more biomarkers in the sample is achieved.

The present invention provides a step of comparing the quantity of one or more NE biomarkers to normal levels to determine the injury, disease, or repair of the subject. It is appreciated that selection of the NE biomarkers or even additional biomarkers allows one to identify the types of cells implicated in an abnormal organ or physical condition as well as the nature of cell death in the case of an axonal injury marker. The practice of an inventive process provides a test which can help a physician determine suitable therapeutics to administer for optimal benefit of the subject. While the neural data provided in the examples herein are provided with respect to a full spectrum of TBI, neurotoxicity, and neuronal cell death, it is appreciated that these results are applicable to other aforementioned forms of injury, disease, or repair. As is shown in the subsequently provided example data, a gender difference is unexpectedly noted in abnormal subject injury, disease, or repair.

The results of such a test using an in vitro diagnostic device can help a physician determine whether the administration of a particular therapeutic or treatment regimen may be effective and provide a rapid clinical intervention to the injury or disorder to enhance a patient's recovery.

It is appreciated that other reagents such as assay grade water, buffering agents, membranes, assay plates, secondary antibodies or aptamers, salts, and other ancillary reagents are available from vendors known to those of skill in the art.

Methods involving conventional biological techniques are described herein. Such techniques are generally known in the art and are described in detail in methodology treatises such as Molecular Cloning: A Laboratory Manual, 2nd ed., vol. 1-3, ed. Sambrook et al., Cold Spring Harbor Laboratory Press, Cold Spring Harbor, N.Y., 1989; and Current Protocols in Molecular Biology, ed. Ausubel et al., Greene Publishing and Wiley-Interscience, New York, 1992 (with periodic updates). Immunological methods (e.g., preparation of antigen-specific antibodies, immunoprecipitation, and immunoblotting) are described, e.g., in Current Protocols in Immunology, ed. Coligan et al., John Wiley & Sons, New York, 1991; and Methods of Immunological Analysis, ed. Masseyeff et al., John Wiley & Sons, New York, 1992.

### 4. Examples

This invention is not limited to the particular processes, compositions, or methodologies described, as these may vary. The terminology used in the description is for the purpose of describing the particular versions or embodiments only, and is not intended to limit the scope of the present invention which will be limited only by the appended claims. Although any methods and materials similar or equivalent to those described herein can be used in the practice or testing of embodiments of the present invention, the preferred methods, devices, and materials are now described. All publications mentioned herein, are incorporated by reference in their entirety; nothing herein is to be construed as an admission that the invention is not entitled to antedate such disclosure by virtue of prior invention.

### Example 1: Methods

Study of HIE neonates: Human neonates meeting the inclusion/exclusion criteria in Table 2, below, are eligible for enrollment. Hypothermia treatment is performed on those neonates who meet the HIE criteria according to the hypothermia protocol of the FN3 that is derived from the NICHD trial (Table 2). Each participating site in the study has a gold standard Sarnat evaluator. Members of FN3 have been trained on the Sarnat evaluation for neonates with HIE using the NICHD approved examination. Each site investigator is required to retake the examination course from the NICHD yearly during the grant period.

Currently, hypothermia management is designed to include standardized systemic supportive care protocols (including ionotrope selection and dosing, fluid volumes, targeted glucose ranges), a centralized data repository for capturing patient demographics (REDCap), standardized MRI result reporting, developmental follow-up time line, and a serum sample repository. The data recorded in REDCap also allows analysis of the presence of a sentinel event such as a placental abruption.

TABLE 2
Inclusion and Exclusion Criteria

| Inclusion Criteria | Exclusion Criteria |
|---|---|
| 1. Gestational age ≥ 36 weeks and birth weight ≥ 1.8 kg | 1. Lethal chromosomal |
| and ≤6 hours from insult | abnormalities, or |
| 2. Seizures or 3 of the 6 clinical signs of HIE | 2. Severe intrauterine growth |
| 3. One or more of predictors of severe HIE: | restriction, or |
| a. pH ≤ 7.0 with base deficit ≥ 16 on arterial blood | 3. Significant intracranial |
| gas, | hemorrhage (grade III or |
| b. pH 7.01-7.15, base deficit 10-15.9 or no blood gas | intraparenchymal echodensity |
| available and acute perinatal event (cord prolapse, heart | (grade IV)), or |
| rate decelerations, uterine rupture, and either | 4. Sepsis evaluation with clinical |
| c. Apgar score ≤ 5 at 10 minutes or | signs and symptoms consistent |
| d. assisted ventilation at birth for ≥10 minutes | with encephalitis. |

Timing of blood samples in neonates with moderate or severe HIE who qualify for hypothermia treatment: Serum samples were collected from neonates with HIE at 0-6, 12, 24, 48 and 96 hours of age. Neonates with HIE who meet the criteria for hypothermia treatment are enrolled and stratified using the inclusion/exclusion criteria in Table 2. The exact time of birth and sample collection is recorded. Since HIE patients should be >1.8 kg at birth, the total blood sampling is a maximum 3.3 mL/kg for the total study time (within acceptable IRB limits). These inclusion and exclusion criteria match those in the published major hypothermic treatment trials, allowing for comparisons between this study and those trials. Blood samples are drawn via venipuncture, collected in Serum Separator Tubes (Quest Diagnostics, N.J.), left to clot for 30-60 minutes, and centrifuged at 1,500 rpm for 15 minutes before storage at −80° C. and shipment on dry ice to a central repository.

Protein biomarker assays: For the 4 biomarkers (GFAP, UCH-L1, T-Tau) analyzed, the Quanterix Simoa™ N4PB kit is used to measure GFAP, UCH-L1, T-Tau, and NF-L serum concentrations run simultaneously in a multiplex format in the same reaction well for each sample using the Quanterix™ Neurology 4 Plex kit (N4PB prerelease version), according to the manufacturer's instructions (provided online at quanterix.com. Assay methodology is described in detail in Korely et al., 2018.

miRNA biomarker assays: Table 3, below, shows representative demographics of the study population by cohort assignment: HIE (n=40) and control (n=9), miRNA quantitation in serum samples from these subjects are performed with the FirePlex™ barcoded particle multiples PCR platform (Abcam™). MicroRNAs are screened using a commercial neurological kit (abcam #ab218342) according to the manufacturer's protocol. The steps include serum lysate, hybridization, probe labeling, PCR and data report. Briefly, 40 μL serum sample and 40 μL Lysis Mix are incubated at 60° C. for 45 minutes to obtain digested serum. Then, 35 μL FirePlex® Particles, 25 μL hybridization buffer and digested serum are mixed in a filter plate followed by rinsing with Rinse Buffer. Seventy-five microliters Labeling Mix then is added and allowed to shake at room temperature for 60 minutes. After rinsing, 110 μL RNase-free water is added and shaken at 55° C. for 30 minutes. A vacuum then is applied to elute the solution and then 30 μl eluent is transferred to the PCR plate. Two to twenty microliters of PCR Master Mix is added to initiate the PCR amplification. To read the signals, 20 μL PCR product and 60 μL Hyb Buffer are mixed at 37° C. for 30 minutes. After rinsing, reporter Mix and run buffer are added. The plate is read on a Guava™ 6HT flow cytometer with a 96-well plate autosampler component. The results are analyzed using online FirePlex® Analysis Workbench Software.

Biomarker Analysis-Enzyme-linked immunosorbent assay (ELISA): Measurement of GFAP, UCH-L1, NFL and Tau concentrations were measured using the same batch of reagents by investigators blinded to clinical data using SIOMA neuro 4 plex kit in SR-X immunoassay analyzer (Quanterix Corp, Boston, Mass., USA), which runs ultrasensitive paramagnetic bead-based enzyme-linked immunosorbent assays.
MRI scoring: MRI were performed at either 3-5 (n=36) days of age following rewarming or 7-12 days of age (n=5) if the neonates were not stable enough for transport for MRI at 3-4 days of age. Neonates were imaged on a Siemens Magnetom Verio 3T scanner (Siemens, Malvern, Pa.) at UF Health Gainesville. Analysis focused on the Thweighted, T2-weighted, and diffusion-weighted imaging (DWI) abnormalities. Two blinded subspecialty board-certified neuroradiologists with over 10 years of experience in neonatal imaging interpreted all the MRI images using the Barkovich scoring system (21). The Barkovich scoring system scores injury in different brain regions using a scale with increasing values representing worsening injury. Individual brain regions scored included the basal ganglia (0-4), the watershed, cortex/white matter (0-5) and combined basal ganglia/watershed (0-4). Brain injury was stratified according to location into three groups: basal ganglia, watershed and combined basal ganglia/watershed. Infants with scores of 0-2 in any region were categorized as no/mild injury and infants with scores equal to or greater than 3 in any region were coded as moderate/severe injury.
Outcome assessment (Bayley III Testing at 17-24 months of age): All patients with NE are followed in our Early Developmental Assessment clinic and receive a Bayley III exam as part of their routine clinical care. We analyzed 20 subjects that had available biomarker data and Bayley III exams ranging from 17-24 months of age with an average of 20 months of age. Individual developmental domains on the Bayley III including motor, cognitive and language were analyzed. The analysis was performed using normal-theory simple linear regressions (linear models, LM) to relate the biomarker concentrations and the Bayley domain scores. Logistic regressions with one covariate (generalized linear models, GLM) were used to relate the binary responses to the biomarkers as a good outcome with Bayley domain scores of 85 or greater or a poor outcome, defined as a Bayley score of less than 85.
Data Management and Statistical Analysis: Study data was captured through REDCap software (22) and stored within the Academic Information Systems and Support servers within the UF Health Science Center Secure Enterprise Data Center, which is comprised of redundant, high availability infrastructure components with secure data storage and scheduled maintenance. One-way analysis of variance was used to assess whether the mean concentrations differ among the groups for each of the time points. All biomarker values were logarithmically transformed to attain normal distribution. Descriptive statistics (i.e. mean, median, standard deviation) were calculated for continuous variables. Mann-Whitney U and Kruskal-Wallis tests were conducted to assess differences between groups for continuous variables. Frequencies and percentages were determined for categorical variables. Chi-square with Fisher's exact test were used to determine associations for categorical variables. The accuracy of biomarker levels to differentiate between good and poor outcome was evaluated by the receiver operating characteristic (ROC) analysis. All tests were two tailed, with a significance level set at 5% and the analysis was performed in SPSS version 21.0.

Trajectory describes the course of a measured variable over time. It identifies groups of individuals following similar progressions of some phenomenon over time and estimates the effects of covariates not only on trajectory shape, but also group membership (23). Trajectory analyses were conducted using SAS 9.4, R 3.4, and R studio 1.0 statistical software. Using the LCMM 1.7.8 package in R (https://arxiv.org/pdf/1503.00890.pdf), we used unconditional LCMM for ordinal data to model the combination of four biomarkers' trajectories over time and to classify patients into distinct latent trajectory classes. The only variables used to infer latent class were subject, combined biomarker levels (ratios to the 0-6h data point), and time. We used unconditional LCMM (instead of conditional LCMM) because our primary aim was to describe the “raw” latent biomarker trajectories in the population without imposing any conditions or predictors on the model. Our secondary aim was to then explore predictors of these unconditional trajectories. LCMM assumes that the population is heterogeneous and is divided into distinct groups, with each group having its own trajectory of combined biomarker levels versus time. LCMM, like other likelihood-based methods, can analyze data with missing observations. As long as missing observations are missing in a way that depends only on observed values, then the estimates will be unbiased. Starting with a one-class model, we fitted models with increasing numbers of classes until we reached the inflection point of the Akaike information criterion (AIC). The AIC is a way to identify the point at which the benefits of improved model fit are outweighed by the cost of model complexity. We additionally examined the log likelihood, a measure of goodness of model fit regardless of model complexity, and the Bayesian information criteria (BIC). The BIC is similar to the AIC but has a slightly different threshold such that increased model complexity is penalized more heavily than it is in the AIC, generally resulting in an inflection point at a less complex model.

TABLE 3
Demographics of Study Population by Cohort Assignment.

| Patient | Total | | Cohort 1 | | Cohort 2 | |
|---|---|---|---|---|---|---|
| Demographics | (n = 49) | | (n = 9) | | (n = 40) | |
| Gender (%) | | | | | | |
| Female | 15 | (30.6) | 14 | (35) | 1 | (12.5) |
| Male | 34 | (69.4) | 26 | (65) | 8 | (87.5) |
| Race (%) | | | | | | |
| White | 29 | (59.2) | 24 | (60) | 5 | (55.6) |
| Black | 12 | (24.5) | 9 | (22.5) | 3 | (33.3) |
| Asian | 1 | (2.4) | 1 | (2.5) | 0 | (0) |
| Unknown | 5 | (10.2) | 4 | (10) | 1 | (11.1) |
| Other | 2 | (4.1) | 2 | (5) | 0 | (0) |

| | | | |
|---|---|---|---|
| Birth Weight | 3340 ± 791.07 | 3336 ± 839.9 | 3361 ± 560.5 |
| (g)* | | | |

*Birth weight: values are means ± SD.
Cohort 1 = HIE; Cohort 2 = controls.

### Example 2. Temporal Profile of Neuroprotein Biomarkers

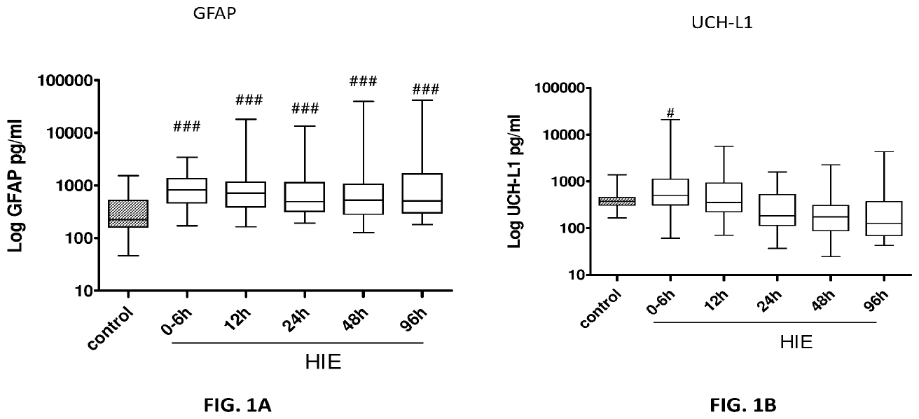

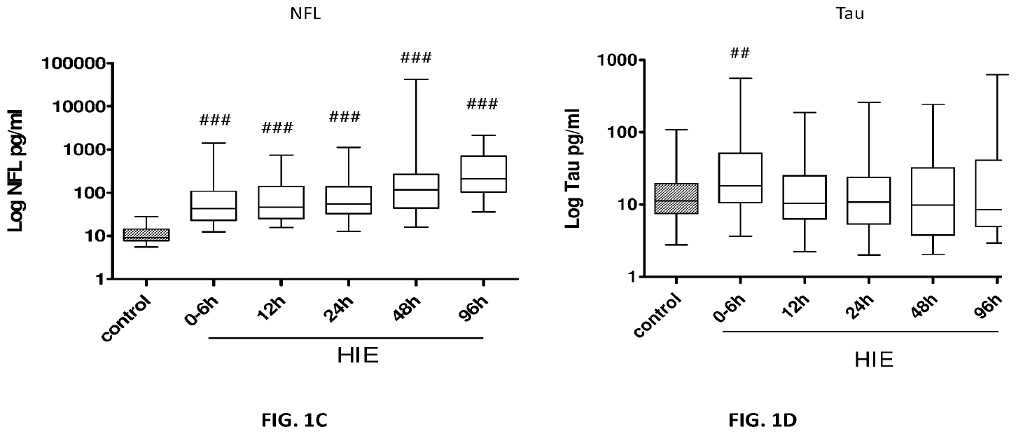

Different biomarkers have different temporal elevation profiles in HIE. The protein biomarker levels in blood at different time intervals predict HIE-related brain injury detectable by MRI at 3 days of life in these patients. According to embodiments of this invention, four brain biomarkers that have different temporal elevation profile (UCH-L1, GFAP, NF-L, Tau) were identified. See FIG. 1.

### Example 3. Correlation of Protein Biomarkers with Injury Severity

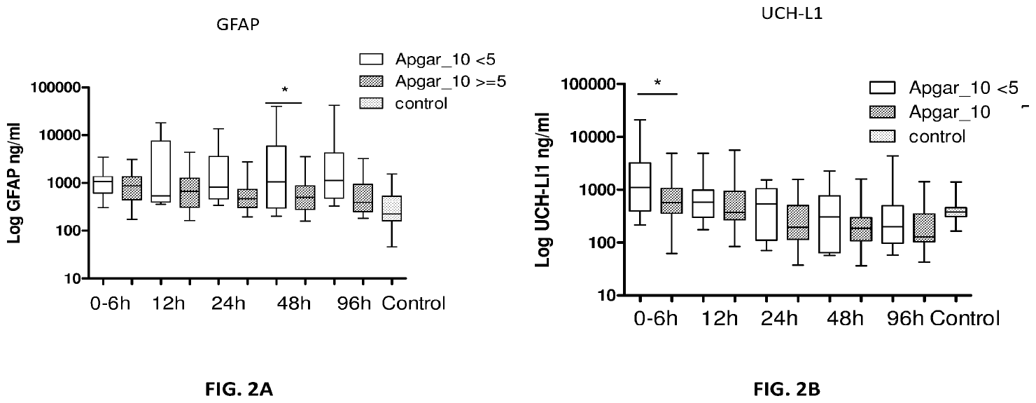

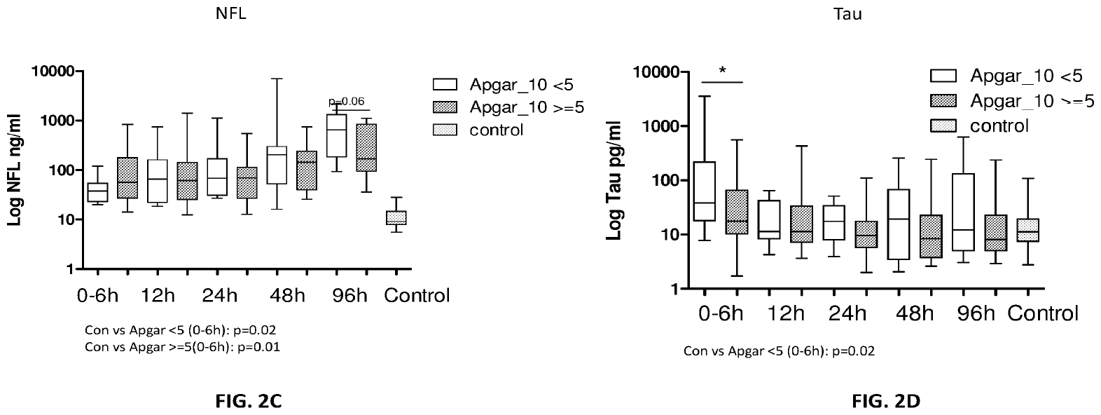

FIG. 2 presents data on the level of the indicated biomarkers in patients with an Apgar 10 score of less than 5, greater than or equal to 5, and control non-HIE patients. The Apgar severity score was assessed at 10 minutes after birth. The higher severity HIE group (Apgar <5) have higher 0- to 6-hour UCH-L1 and Tau biomarker levels, and 48-hour GFAP levels and 96-hour NF-L levels than their counterparts in the lower severity group (Apgar ≥5). Several of the biomarker levels in Apgar <5 are also high than normal controls. These data indicate that acute blood-based protein biomarker levels at 0-96 hours after birth are correlated with injury severity (Apgar score 1-10). FIG. 2. Acute blood-based protein biomarkers (0-96 h) are correlated with injury severity (Apgar score 1-10). Higher severity HIE group (Apgar <5) have higher 0-6 UCH-L1 and Tau, biomarker levels, and 48h GFAP levels and 96 h NF-L levels than their counterparts in the lower severity group (Apgar ≥5). Apgar severity score was assessed at 10 min. Several biomarker levels in Apgar <5 are also high than normal controls.

Table 4, below, shows the Apgar scoring system (Apgar severity score at 10 minutes after birth.

TABLE 4
Apgar Scoring System.

| Indicator | | 0 Points | 1 Point | 2 Points |
|---|---|---|---|---|
| A | Activity | Absent | Flexed limbs | Active |
| | (muscle tone) | | | |
| P | Pulse | Absent | <100 bpm | >100 bpm |
| G | Grimace | Floppy | Minimal | Prompt |
| | (reflex | | response to | response to |
| | irritability) | | stimulation | stimulation |
| A | Appearance | Blue/Pale | Pink | Pink |
| | (skin color) | | body/Blue | |
| | | | extremities | |
| R | Respiration | Absent | Slow and | Vigorous cry |
| | | | irregular | |

### Example 4. Correlation of Protein Biomarker Levels to Brain Injury Score

Biomarker levels were measured at different times after birth as indicated in FIG. 3 and correlated with the brain injury score for basal ganglia MRI, watershed MRI, and thalamus/basal ganglia/cortex MRI. Scores for the different brain regions are shown in FIG. 3, correlated with GFAP, UCH-L1, NF-L and Tau. These data show the correlation between long-lasting higher levels of GFAP and abnormal brain injury shown by MRI. Protein biomarkers level in blood at different time intervals are predictive of HIE-related brain injury detectable by MRI at 3 day of life. Using a composite score for all 4 markers produces a more robust prediction of MRI detectable brain injury. The MRI scores are between 0-5. 0 is no-injury, 5 is the series injury. The score are obtained by the specialist.

### Example 5. Correlation of Protein Biomarker Levels to SARNAT Score

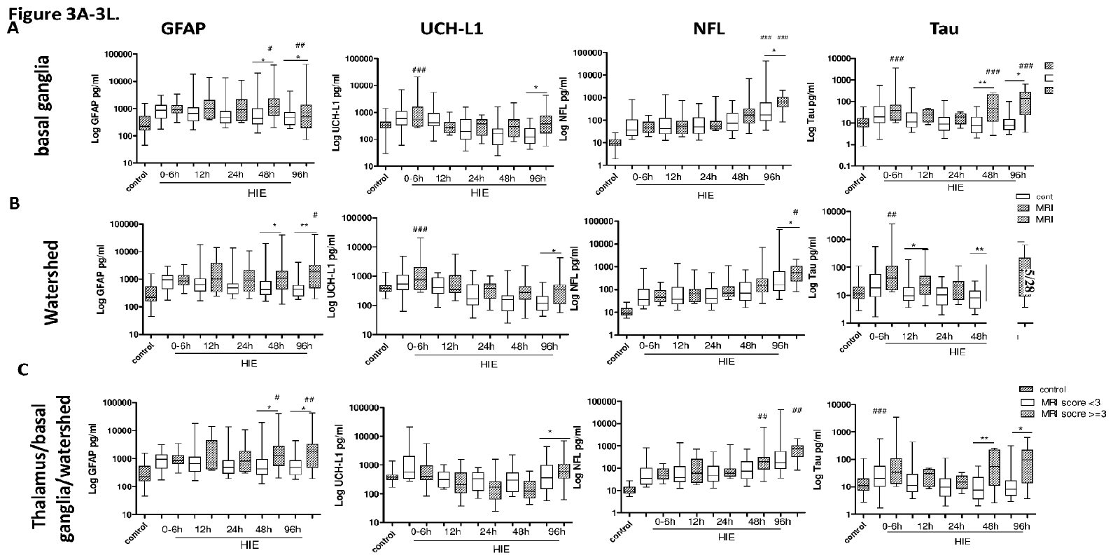

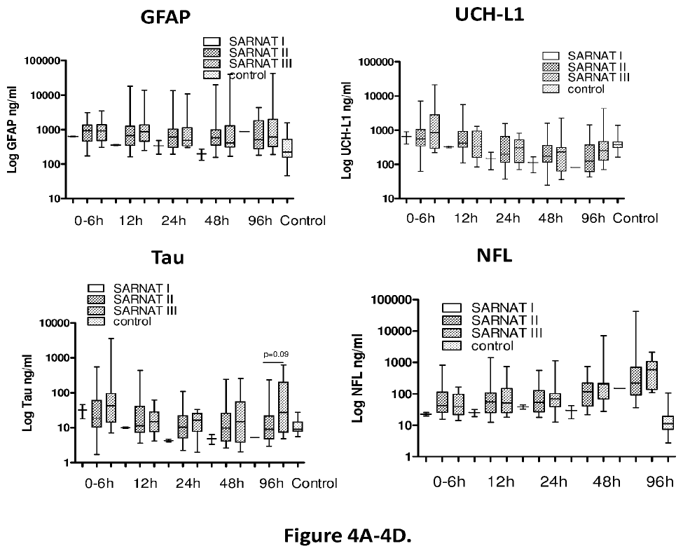

Biomarker levels were measured at different times after birth as indicated in FIG. 4 and correlated with the SARNAT score for HIE staging. Stage III is the most severe; Stage II moderate; and stage I is mild. The data in FIG. 4 show that protein biomarkers levels in blood at different time intervals (e.g. Tau) are correlated to the SARNAT score for HIE staging. In addition, the four protein biomarkers measured at more than one time intervals have advantages and unique clinical utilities. Among these four protein biomarkers, GFAP and NFL are later marker to distinguish between control and patient at stage a particular stage number, while UCHL1 and Tau are early markers that can show differences at less than 6 hours after birth.

Table 5, below shows the SARNAT scoring system.

TABLE 5
SARNAT Scoring System.

| | Level of Consciousness | | |
|---|---|---|---|
| | Stage 1 | Stage 2 | Stage 3 |
| | (mild) | (moderate) | (severe) |
| | hyperalert | lethargic/obtunded | stuperous |
| Neuromuscular Control | | | |
| muscular tone | Normal | Mild hypotonia | Flaccid |
| posture | Mild distal | Strong distal | Intermittent |
| stretch | flexion | flexion | decerebration |
| segmental myoclonus | Overactive | Overactive | Decreased/absent |
| | Present | Present | Absent |
| Complex reflexes | | | |
| suck | Weak | Weak/absent | Absent |
| moro | Strong | Weak | Absent |
| oculovestibular | Normal | Overactive | Weak/absent |
| tonic neck | Slight | Strong | Absent |
| Autonomic function | | | |
| pupils | Mydriasis | Miosis | Variable |
| heart rate | Tachycardia | Bradycardia | Variable |
| bronchial/salivary | Sparse | Profuse | variable |
| secretions | Normal/decreased | Increased/diarrhea | |
| gastrointestinal | | | |
| motility | | | |
| Seizures | None | Common/focal or | Uncommon |
| | | multifocal | |
| EEG | Normal/decreased | Early low voltage | Early periodic |
| (electroencephalogram) | | continuous delta | pattern with |
| | | and theta, later | isopotential |
| | | periodic seizures | phases, later |
| | | focal 1-1.5 Hz | isopotential |
| | | spike wave | |
| Duration | <24 hours | 2-14 days | Hours-weeks |

### Example 6. Correlation of Protein Biomarker Levels to Other HIE Assessment Tools

Biomarker levels were measured at different times after birth as indicated in FIG. 5 and correlated with other HIE assessment tools (pH, blood lactate levels and sentinel event) as discussed above. These data show that protein biomarker levels in blood at different time intervals are correlated to other HIE assessment tools. See FIG. 5 (pH>7, lactate>7). The patient shows sentinel event indicating severe HIE, and compared to severe HIE, mild HIE presents with lower biomarker levels.

### Example 7. Correlation of Protein Biomarker Levels to Bayley Outcome Score

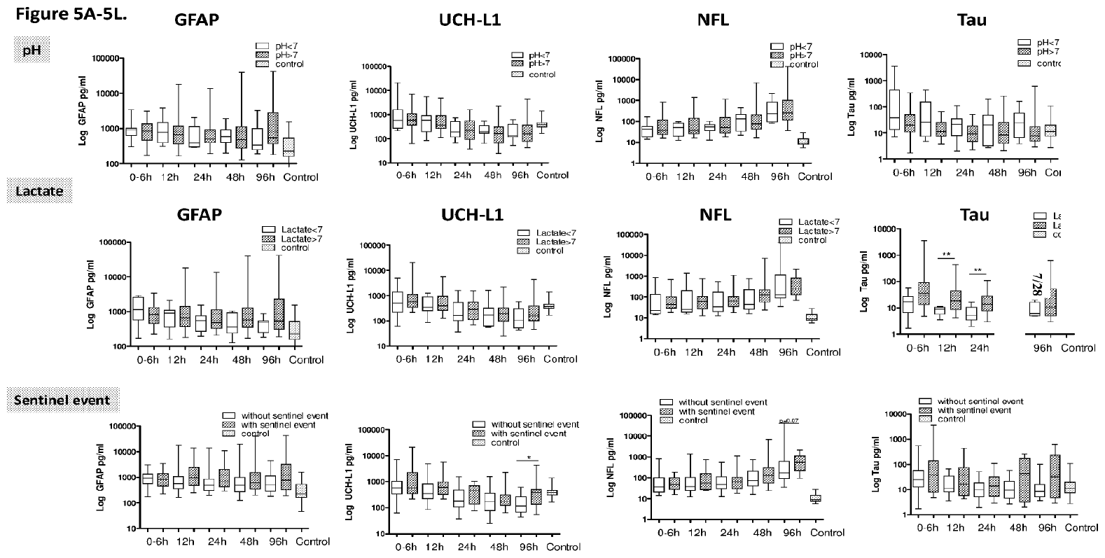

Biomarker levels were measured at different times after birth as indicated in FIG. 6 and correlated with Bayley Outcome Scores assessed at 18-24 months of age, including cognitive function scores, language scores and motor function scores. Higher Bayley subscores indicated better outcome for the patient. FIG. 6. MRI scores range 0-5 from no injury, mild injury to severe injury. Compared to severe HIE, the mild HIE shows lower biomarker levels. Biomarker levels at 48 and 96 hours can differ in mild injury, moderate injury, and severe injury.

### Example 8. Area Under ROC Curve Performance

See Table 6, Table 7, and Table 8, below, for area under the receiver operating characteristic (ROC) curve performance concentrations for prognosticating 18-24 month Bayley Outcome Score, including cognitive function score (Table 6), language score (Table 7), and motor function score Table 8) using biomarker concentrations. These data present 12, 24 and 48 hour GFAP levels, 24 and 96 hour UCHL1 levels and 96 hour Tau levels which could predicted cognitive function. The combination of 24 hour GFAP and UCHL1 or the combination of 24 hour GFAP and 96 hour Tau promoted the performance. GFAP at 48 hours, the combination of 48 hour GFAP and 48 hour UCHL1, the combination of 48 hour GFAP and 48 hour Tau and 48 hour GFAP and 96 hour NFL predicted motor function outcome. GFAP levels at 12, 24 and 28 hours, UCHL1 levels at less than 6 hours, and at 24 and 96 hours, Tau level at 96 hours and NFL levels at 96 hours predicted language function. The combination of 24 hour GFAP and 24 hour UCHL1, 24 hour GFAP and 96 hour Tau or 24 hour GFAP and 96 hour NFL promoted the performance.

TABLE 6
Area under ROC Curve Performance, Cognitive Function.

| | | | | Asymptotic 95% | |
|---|---|---|---|---|---|
| | | | | confidence interval | |
| | | Standard | Asymptotic | Lower | Upper |
| Test Result Variable(s) | Area | error^{a} | Sig.^{b} | bound | bound |
| GFAP_12 | 0.800 | 0.112 | 0.046 | 0.581 | 1.000 |
| GFAP_24 | 0.788 | 0.156 | 0.055 | 0.482 | 1.000 |
| GFAP_48 | 0.812 | 0.104 | 0.038 | 0.607 | 1.000 |
| UCHL1_24 | 0.788 | 0.115 | 0.055 | 0.563 | 1.000 |
| UCHL1_96 | 0.906 | 0.065 | 0.007 | 0.778 | 1.000 |
| Tau_96 | 0.835 | 0.096 | 0.026 | 0.648 | 1.000 |
| GFAP_24 + UCHL1_24 | 0.814 | 0.126 | 0.025 | 0.567 | 1.000 |
| GFAP_24 + Tau96 | 0.835 | 0.119 | 0.026 | 0.603 | 1.000 |

TABLE 7
Area under ROC Curve Performance, Motor Function.

| | | | | Asymptotic 95% | |
|---|---|---|---|---|---|
| | | | | confidence interval | |
| | | Standard | Asymptotic | Lower | Upper |
| Test Result Variable(s) | Area | error | Sig. | bound | bound |
| GFAP_48 | 0.756 | 0.096 | 0.027 | 0.569 | 0.943 |
| GFAP_48 + UCHL48 | 0.762 | 0.095 | 0.024 | 0.577 | 0.947 |
| GFAP_48 + Tau48 | 0.786 | 0.090 | 0.014 | 0.608 | 0.963 |
| GFAP_48 + NFL96 | 0.774 | 0.093 | 0.018 | 0.591 | 0.956 |

TABLE 8
Area under ROC Curve Performance, Language Function.

| | | | | Asymptotic 95% | |
|---|---|---|---|---|---|
| | | | | confidence interval | |
| | | Standard | Asymptotic | Lower | Upper |
| Test Result Variable(s) | Area | error^{a} | Sig.^{b} | bound | bound |
| GFAP_12 | 0.889 | 0.088 | 0.006 | 0.716 | 1.000 |
| GFAP_24 | 0.844 | 0.133 | 0.016 | 0.585 | 1.000 |
| GFAP_48 | 0.744 | 0.117 | 0.087 | 0.515 | 0.974 |
| UCHL1_0-6 | 0.733 | 0.140 | 0.102 | 0.459 | 1.000 |
| UCHL1_24 | 0.856 | 0.092 | 0.013 | 0.676 | 1.000 |
| UCHL1_96 | 0.889 | 0.072 | 0.006 | 0.748 | 1.000 |
| Tau_96 | 0.756 | 0.125 | 0.073 | 0.510 | 1.000 |
| NFL_96 | 0.800 | 0.113 | 0.036 | 0.579 | 1.000 |
| GFAP_24 + UCHL1_24 | 0.844 | 0.123 | 0.016 | 0.603 | 1.000 |
| GFAP_24 + Tau96 | 0.778 | 0.126 | 0.052 | 0.531 | 1.000 |
| GFAP_24 + NFL96 | 0.833 | 0.102 | 0.020 | 0.634 | 1.000 |

### Example 9. Machine-Learning Based Trajectory Analysis

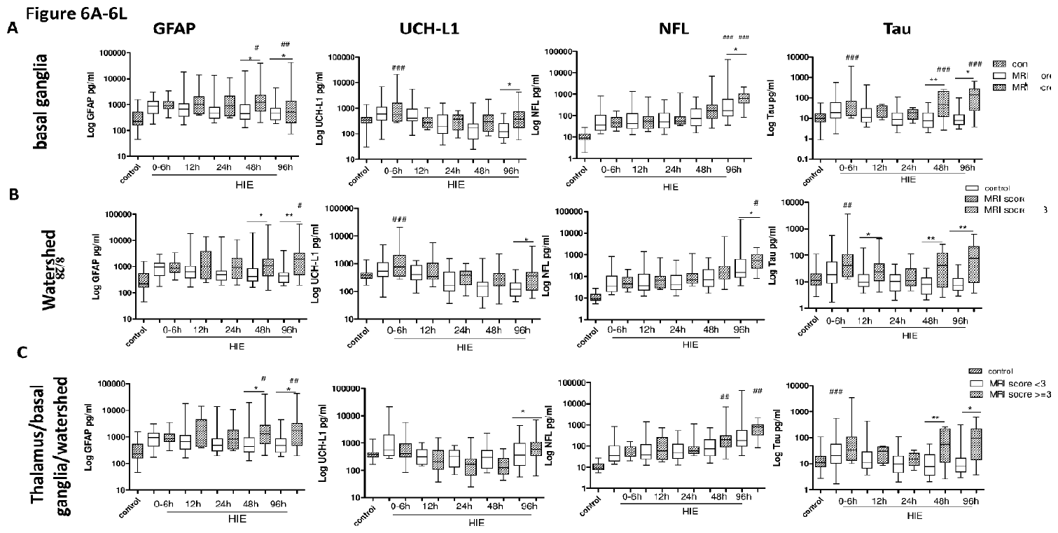

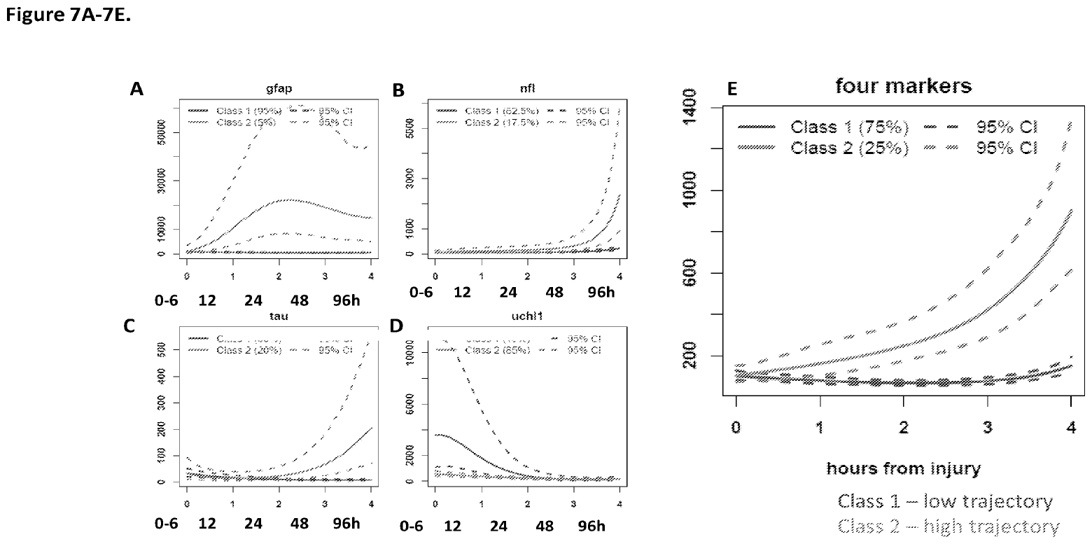

FIG. 7 shows that machine-learning based trajectory analysis identified that there are subgroups of HIE patients with different biomarker trajectories for GFAP (FIG. 7A), NF-L (FIG. 7B), Tau (FIG. 7C), and UCH-L1 (FIG. 7D) and the composite four biomarker levels (FIG. 7E). High and low trajectory groups were identified. In addition, comparing data using single biomarkers or a combination of the four protein biomarkers, different HIE patients can be categorized into different trajectory patterns or classes (high or low). This can be relevant to precision medicine-based patient treatment, management and care.

A data-driven trajectory analysis is a specialized application for capturing the multiple distinct clinical trajectories without imposing a pre-conceived and possible inadequate, stratification system. Data-driven analysis that identify groups of patients with similar trajectories, thus facilitating identification and visualization of multiple distinct outcome trajectories within a single population. This finite mixture modeling determines trends in longitudinally collected data by identifying trajectory groups on a likelihood basis and does not rely solely on mean averages or peak concentrations of biomarkers. A trajectory analysis using latent class mixed models (LCMM) was performed. We assessed the temporal biomarker profiles for HIE prognosis by applying a group-based trajectory modeling approach to classify and characterize patients based on their biomarkers trajectories during the first 96 h of life. Individual biomarker or combination of four markers (GFAP+UCH-L1+NFL+Tau) was analyzed. For combination markers, groups were created using relative biomarker concentration changes from baseline (0-6h) at each sample collection time point (expressed as a ratio). Due to the non-normal distribution of the ratio values, the data was natural log transformed prior to modeling. Individual patients' biomarker trajectories were independently sorted for best fit into high- or low-trajectory groups. Serum four marker levels in the high group consistently increased and was associated with a poor outcome, while the low group kept a stably low biomarker level and was associated with a good outcome on the Bayley at 18-24 months of age.

### Example 10. Four-Biomarker Composite Score Correlation with Clinical Outcome

See Table 9, below for patient biomarker group-based trajectory modeling (TRAJ) class and Bayley scores as indicated. Total patients n=29. Class 1 low biomarker TRAJ: n=20; Class 2 high biomarker TRAJ: n=5.

TABLE 9
Patient Biomarker TRAJ Class and Bayley Scores.

| | Biomarker TRAJ | | | |
|---|---|---|---|---|
| ID | Class | Bayley_cog85 | Bayley_Lang85 | Bayley_Motor85 |
| BP-02 | 1 | 1 | 1 | 2 |
| BP-08 | 1 | 2 | 2 | 1 |
| BP-09 | 1 | 2 | 2 | 2 |
| BP-10 | 1 | 2 | 2 | 2 |
| BP-11 | 1 | 2 | 1 | 2 |
| BP-13 | 1 | 1 | 1 | 1 |
| BP-14 | 1 | 2 | 2 | 2 |
| BP-15 | 1 | 2 | 2 | 2 |
| BP-19 | 1 | 2 | 2 | 1 |
| BP-20 | 1 | 2 | 2 | 2 |
| BP-21 | 1 | 2 | 2 | 2 |
| BP-23 | 1 | 2 | 2 | 1 |
| BP-27 | 1 | 2 | 2 | 2 |
| BP-28 | 1 | 2 | 2 | 2 |
| BP-31 | 1 | 2 | 2 | 1 |
| BP-32 | 1 | 2 | 2 | 2 |
| BP-33 | 1 | 2 | 2 | 2 |
| BP-35 | 1 | 2 | 2 | 1 |
| BP-36 | 1 | 2 | 2 | 1 |
| BP-37 | 1 | 2 | 2 | 1 |
| BP-04 | 2 | 1 | 1 | 1 |
| BP-05 | 2 | 1 | 1 | 1 |
| BP-12 | 2 | 1 | 1 | 1 |
| BP-16 | 2 | 1 | 1 | 1 |
| BP-39 | 2 | 1 | 1 | 1 |

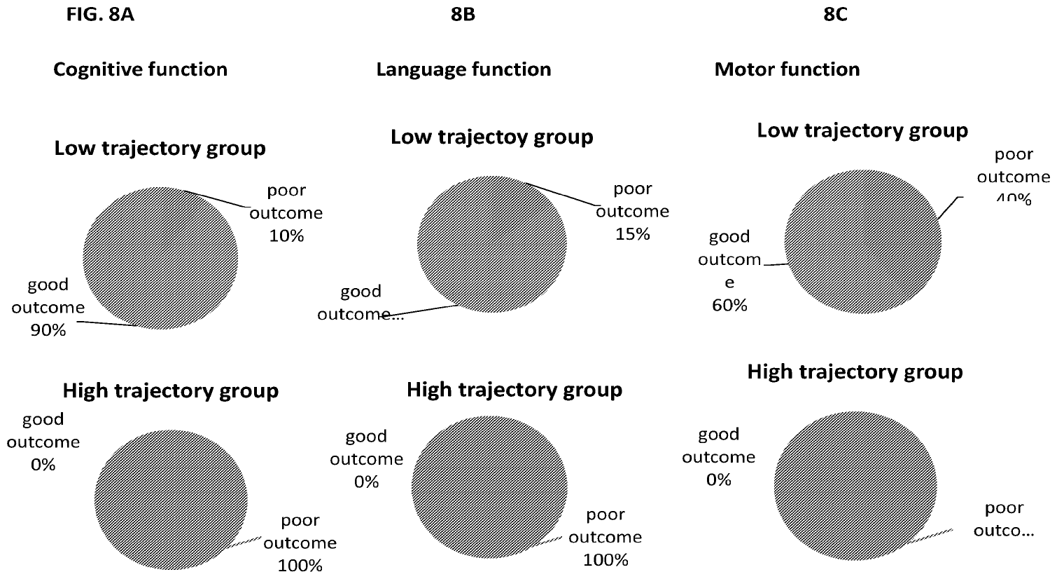

FIG. 8 presents data for the 0-6 hour standardized 4-marker Composite score based trajectory Classes vs. Outcome (Bayley score) at 18-24 month follow-up. The results show that using a composite score for the four protein biomarkers has distinct advantages and clinical utilities with respect to outcome prediction (prognosis). Specifically, for the Bayley Cognitive score (FIG. 8A and Table 10), all 18 patients with a score ≥85 (good outcome) are in the Class 1 Low biomarker TRAJ group; for the Bayley Language score (FIG. 8B and Table 11), all 17 patients with a score ≥85 (good outcome) are in the Class 1 Low biomarker TRAJ group; for the Baylor Motor score (FIG. 8C and Table 12), all 13 patients with score ≥85 are in Class 1 Low biomarker TRAJ group. Thus, the 4-biomarker composite score trajectory-based grouping can predict HIE patient outcome at 18-24 months. Groups were created using relative biomarker concentration changes from baseline (0-6 hours) at each sample collection time point (expressed as a ratio). A data-driven trajectory analysis using latent class mixed models (LCMM) was performed. Due to the non-normal distribution of the ratio values, the data was natural log transformed prior to modeling. Trajectory analysis identified two different TRAJ group profiles: high and low combination of combined four serum biomarkers (GFAP+UCH-L1+NFL+Tau). Serum four marker levels in the high group consistently increased and was associated with a poor outcome, while the low group kept a stably low biomarker level and was associated with a good outcome on the Bayley at 18-24 months of age.

TABLE 10
Cognitive Function Baylor Score (FIG. 8A).

| | |
|---|---|
| Odds ratio | 2.5714 |
| 95% CI: | 0.9142 to 7.2330 |
| z statistic | 1.790 |
| Significance level | P = 0.0735 |

TABLE 11
Language Function Baylor Score (FIG. 8B).

| | |
|---|---|
| Odds ratio | 2.1250 |
| 95% CI: | 0.7765 to 5.8154 |
| z statistic | 1.468 |
| Significance level | P = 0.1422 |

TABLE 12
Motor Function Baylor Score (FIG. 8C).

| | |
|---|---|
| Odds ratio | 0.9231 |
| 95% CI: | 0.3532 to 2.4125 |
| z statistic | 0.163 |
| Significance level | P = 0.8703 |

TABLE 13
Targeted miRNA panel for HIE.

| | | | | | | | | Physiology | |
|---|---|---|---|---|---|---|---|---|---|
| | *p-value | 0-6 hour | 0-6 hour | 48 hour | 48 hour | Control | Control | and/or | CNS |
| Probe | (ANOVA) | mean* | SD | Mean | SD | Mean | SD | Pathology | Location |
| hsa-mir- | 1.41E−09 | 2572.03 | 1338.51 | 603.92 | 739.46 | 946.73 | 330.28 | neuronal | neuron |
| 145-5p | | | | | | | | differentiation, | |
| | | | | | | | | gliogenesis/ | |
| | | | | | | | | autophagy | |
| hsa-mir- | 5.34E−09 | 36497.04 | 14271.66 | 65852.64 | 17482.67 | 36673.56 | 4072.02 | axonal | axon |
| 16-5p | | | | | | | | outgrowth | |
| hsa-mir- | 6.49E−07 | 11832.56 | 4551.90 | 21000.07 | 8951.12 | 19123.76 | 3887.88 | angiogenesis | astrocyte |
| 15a-5p | | | | | | | | | |
| hsa-mir- | 1.11E−06 | 24251.30 | 8974.70 | 40926.63 | 12415.12 | 28632.48 | 2732.29 | gliogenesis, | glia/axon |
| 17-5p | | | | | | | | axonal | |
| | | | | | | | | outgrowth, | |
| | | | | | | | | apoptosis | |
| hsa-let- | 7.35E−06 | 2987.37 | 1329.48 | 4444.41 | 2431.05 | 7036.47 | 3865.85 | neuronal | neuron/glia/ |
| 7g-5p | | | | | | | | migration, | astrocyte |
| | | | | | | | | gliogenesis, | |
| | | | | | | | | neuronal | |
| | | | | | | | | differentiation | |
| | | | | | | | | angiogenesis/ | |
| | | | | | | | | autophagy | |
| hsa-mir- | 4.42E−05 | 1863.95 | 1234.09 | 590.07 | 1063.66 | 439.52 | 774.75 | dendritic | neuron/glia |
| 214-3p | | | | | | | | outgrowth, | |
| | | | | | | | | gliogenesis | |
| hsa-mir- | 0.000258 | 93.28 | 108.10 | 124.08 | 85.53 | 391.31 | 467.60 | axonal | axon |
| 338-3p | | | | | | | | outgrowth | |
| hsa-mir- | 0.00142 | 523.91 | 208.08 | 450.50 | 162.46 | 293.57 | 96.26 | dendritic | neuron/ |
| 132-3p | | | | | | | | outgrowth, | synapse |
| | | | | | | | | synaptogenesis | |
| hsa-mir- | 0.00191 | 12190.51 | 2860.88 | 8324.22 | 2917.39 | 8845.06 | 2609.08 | myelination, | myelin |
| 23a-3p | | | | | | | | gliogenesis/ | sheath/glia |
| | | | | | | | | necrosis | |
| hsa-mir- | 0.00831 | 279.72 | 212.44 | 382.61 | 318.50 | 798.11 | 835.27 | dendritic | neuron |
| 26b-5p | | | | | | | | outgrowth, | |
| | | | | | | | | neuronal | |
| | | | | | | | | differentiation | |
| hsa-mir- | 0.00996 | 17408.15 | 4366.53 | 15875.12 | 5287.31 | 11603.90 | 4494.21 | gliogenesis/ | glia |
| 146a-5p | | | | | | | | autophagy | |

*levels provided as pg/ml

### Example 11. Human miRNA in Serum as Biomarkers

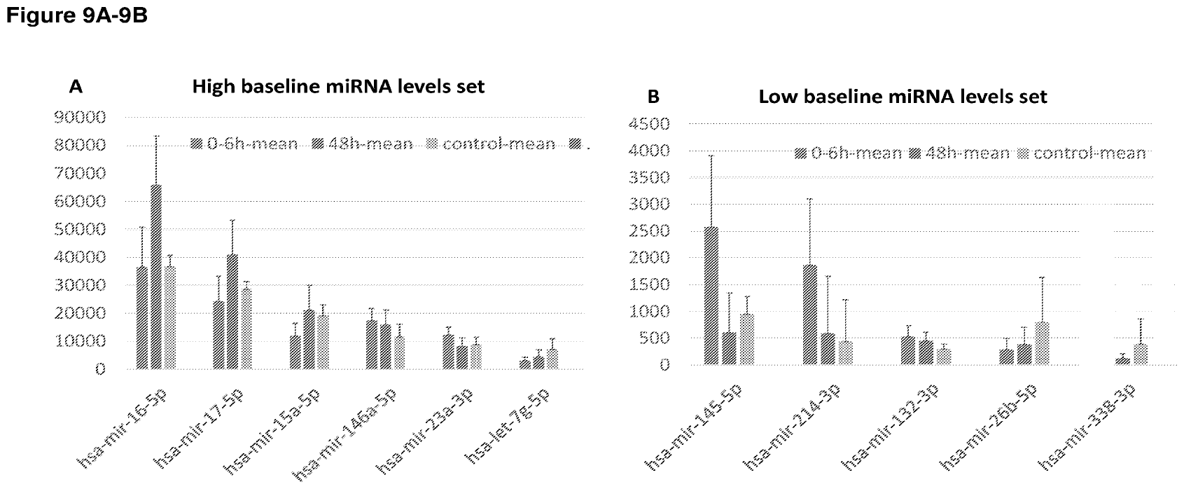

FIG. 9 presents data for a high baseline miRNA set (FIG. 9A) and a low baseline miRNA set (FIG. 9B). These data show that the 11 miRNA from human serum tested have altered levels in HIE (0-6 hours, and/or 48 hours) compared to normal control levels. Of these, 6 out of 34 are in the high baseline miRNA level set. A high level would be greater than 10,000 copies, middle level would be 5000-10000, and low would be less than 5000 copies. Several have are elevated in HIE at only 48 hours (Mir-16, -17), while miR-146a are elevated at both 0-6 hours and 48 hours and mir-23a is elevated at 0-6 hours only. Mir-15 and Let-7g are reduced in the 0-6 hour sample and the 0-6 hour and the 48 hour samples, respectively. Of the low baseline miRNA level set, the HIE samples for mir-145 and mir-214 are elevated at 0-6 hours compared to normal control groups. The mir-132 sample is elevated at both 0-6 hours and 48 hours. Mir-26b and mir-338 are reduced at both 0-6 hours and 48 hours compared to controls. Thus, changes in the levels of these miRNAs are suitable for use as biomarkers for HIE.

See Table 14, below, showing an additional panel of 28 miRNA with altered serum levels when comparing (1) controls (n=9) vs. 0-6 hour HIE (n=40) or (2) controls compared to 48 hour HIE (n=40); p-values are shown. Levels in HIE (0-6 hour), HIE (48 hour) and control, and the SD (standard deviation) are shown. The miRNA assay was performed using a neurology 65 panel (Abcam, Inc). The biomarkers in the table below are those with significant difference at either 0-6h or 48h (P<0.05 compared to controls).

TABLE 14
Targeted miRNA panel for HIE (2^{nd} panel) of 28 significantly altered miRNA.

| | | | | | | | | Physiology | |
|---|---|---|---|---|---|---|---|---|---|
| | p-value | 0-6 hour | 0-6 hour | 48 hour | 48 hour | Control | Control | and/or | |
| probe | (ANOVA) | mean* | SD | mean | SD | mean | SD | Pathology | Expression |
| hsa-mir- | 5.05E−14 | 3018.068 | 1398.57 | 2624.834 | 1055.595 | 9886.1 | 3888.059 | blood brain | expression |
| 150-5p | | | | | | | | barrier | |
| hsa-mir- | 6.38E−11 | 7671.186 | 2683.3 | 4493.401 | 2397.544 | 1958.73 | 1756.332 | apoptosis | endothelial |
| 497-5p | | | | | | | | | cells |
| hsa-mir- | 8.80E−10 | 36147.88 | 13546 | 66776.33 | 19973.94 | 34800.6 | 4576.502 | | |
| 451a | | | | | | | | | |
| hsa-mir- | 1.24E−09 | 676.694 | 371.32 | 854.782 | 461.164 | 2668.45 | 1518.249 | dendritic | neuron/ |
| 29b-3p | | | | | | | | outgrowth, | endothelial |
| | | | | | | | | blood brain | cells |
| | | | | | | | | barrier/apotosis | |
| hsa-mir- | 8.93E−09 | 2109.574 | 2600.86 | 1883.038 | 1635.905 | 407.214 | 299.533 | | |
| 323a-3p | | | | | | | | | |
| hsa-mir- | 9.60E−09 | 7183.702 | 2310.56 | 9571.744 | 2673.137 | 14826.2 | 4426.395 | blood brain | endothelial |
| 107 | | | | | | | | barrier | cells |
| hsa-mir- | 1.00E−08 | 1930.96 | 848.524 | 1633.986 | 711.664 | 4261.01 | 1824.057 | blood brain | endothelial |
| 342-3p | | | | | | | | barrier/ | cells |
| | | | | | | | | excitotoxicity | |
| hsa-mir- | 3.46E−08 | 6142.588 | 6027.04 | 3957.958 | 2565.428 | 1538.76 | 824.388 | | |
| 382-5p | | | | | | | | | |
| hsa-mir- | 3.59E−08 | 28545.545 | 13069.4 | 49290.76 | 15597.88 | 20916.3 | 8331.559 | | |
| 486-5p | | | | | | | | | |
| hsa-mir- | 1.97E−07 | 10902.839 | 3369.37 | 14569.41 | 4171.48 | 20998.5 | 6072.514 | | |
| 103a-3p | | | | | | | | | |
| hsa-mir- | 6.47E−07 | 6623.992 | 2449.67 | 10728.15 | 3682.456 | 12365.2 | 4411.876 | | |
| 15b-5p | | | | | | | | | |
| hsa-mir- | 1.57E−06 | 15818.206 | 6346.18 | 28582.39 | 11592.06 | 19155.1 | 3602.046 | autophagy | |
| 20a-5p | | | | | | | | | |
| hsa-mir- | 6.05E−06 | 333.275 | 216.784 | 466.186 | 284.245 | 1065.77 | 478.103 | | |
| 301a-3p | | | | | | | | | |
| hsa-mir- | 1.04E−05 | 11597.949 | 2697.5 | 8092.663 | 3654.759 | 14455 | 3670.343 | | |
| 24-3p | | | | | | | | | |
| hsa-mir- | 1.31E−05 | 680.172 | 816.443 | 253.661 | 289.485 | 162.262 | 133.811 | dendritic | neuron |
| 134-5p | | | | | | | | outgrowth, | |
| | | | | | | | | neuronal | |
| | | | | | | | | migration, | |
| | | | | | | | | neuron | |
| | | | | | | | | differentiation, | |
| | | | | | | | | neural stem | |
| | | | | | | | | cells | |
| | | | | | | | | proliferation | |
| hsa-mir- | 3.27E−05 | 11378.881 | 3853.13 | 14264.73 | 3930.392 | 21015.9 | 6926.807 | | |
| 191-5p | | | | | | | | | |
| hsa-mir- | 3.77E−05 | 629.583 | 1302.38 | 195.061 | 189.412 | 153.366 | 126.29 | neuronal | neuron |
| 34a-5p | | | | | | | | differentiation, | |
| | | | | | | | | dendritic | |
| | | | | | | | | outgrowth/ | |
| | | | | | | | | apoptosis | |
| hsa-mir- | 5.64E−05 | 78.093 | 44.079 | 36.122 | 25.645 | 165.771 | 58.892 | | |
| 30e-5p | | | | | | | | | |
| hsa-mir- | 0.000102 | 27002.345 | 10241.3 | 42321.81 | 13785.48 | 30181.1 | 3892.658 | | |
| 93-5p | | | | | | | | | |
| hsa-mir- | 0.000194 | 1676.584 | 3265.75 | 81.085 | 1921.99 | 9.358 | 328.531 | | |
| 206 | | | | | | | | | |
| hsa-mir- | 0.000299 | 2085.537 | 784.217 | 2030.336 | 625.162 | 1130.84 | 448.994 | | |
| 151a-3p | | | | | | | | | |
| hsa-mir- | 0.000376 | 5464.16 | 2451.42 | 8775.911 | 4870.004 | 9524.56 | 1707.622 | angiogenesis/ | astrocyte |
| 195-5p | | | | | | | | autophagy | |
| hsa-mir- | 0.000681 | 47.808 | 51.525 | 52.258 | 45.696 | 258.948 | 231.929 | blood brain | endothelial |
| 155-5p | | | | | | | | barrier/ | cells |
| | | | | | | | | autophagy | |
| hsa-mir- | 0.000776 | 1770.715 | 968.616 | 1706.095 | 664.602 | 981.92 | 324.945 | | |
| 328-3p | | | | | | | | | |
| hsa-mir- | 0.000962 | 618.345 | 2357 | 204.096 | 458.312 | 32.283 | 326.003 | | |
| 885-5p | | | | | | | | | |
| hsa-mir- | 0.00113 | 416.164 | 520.712 | 979.786 | 1019.469 | 465.978 | 428.345 | | |
| 7-5p | | | | | | | | | |
| hsa-mir- | 0.00198 | 160.708 | 110.04 | 162.742 | 108.332 | 435.615 | 555.042 | blood brain | endothelial |
| 142-3p | | | | | | | | barrier | cells |
| hsa-mir- | 0.0181 | 196.331 | 85.173 | 240.838 | 152.447 | 371.409 | 313.772 | | |
| 98-5p | | | | | | | | | |

*levels provided as pg/ml

### Example 12. MiRNA Biomarkers

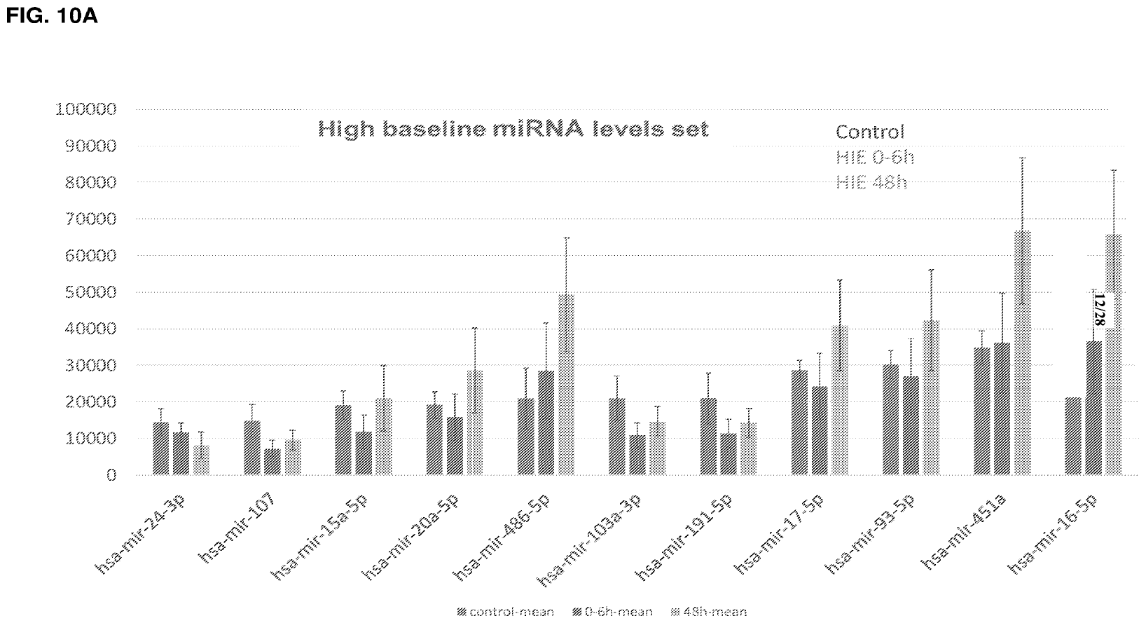

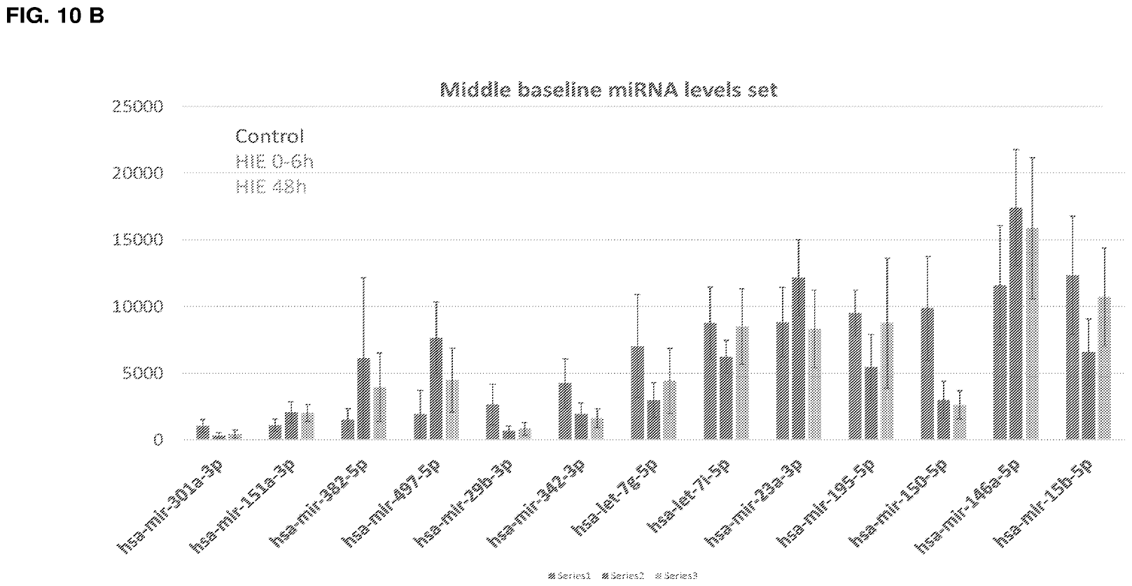

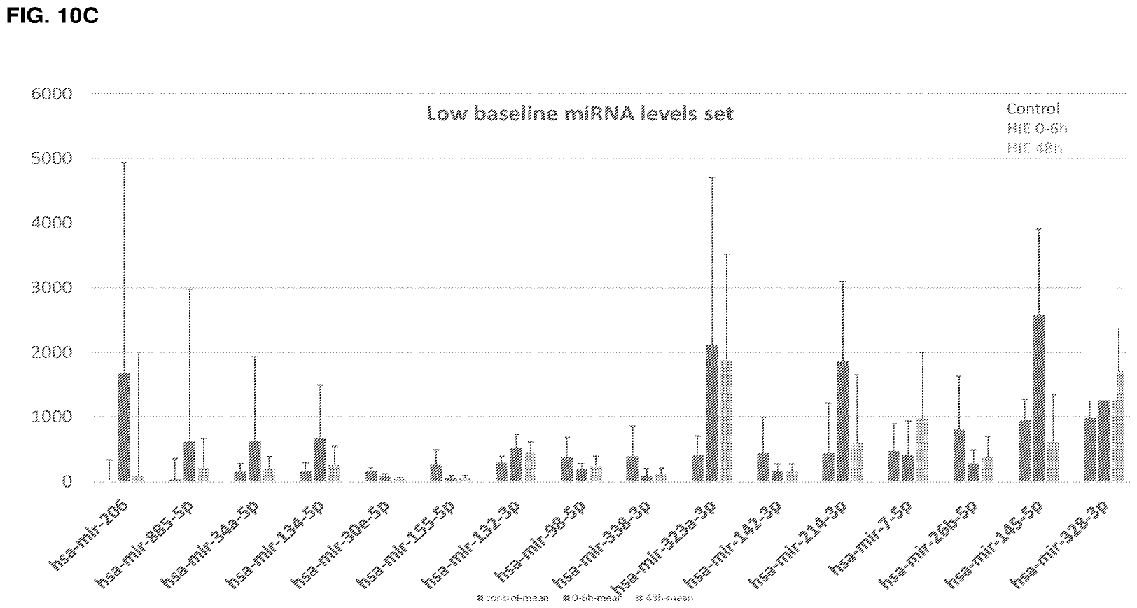

FIG. 10 presents data for high (FIG. 10A), middle (FIG. 10B), and low (FIG. 10C) baseline miRNA levels. FIG. 10A shows data for the 11 human miRNA in serum which were identified as having high baseline levels and having altered levels in HIE (0-6 hour, and/or 48 hours) compared to normal control levels. FIG. 10B shows data for the 16 human miRNA in serum which were identified as having moderate (middle) baseline levels and having altered levels in HIE (0-6 hour, and/or 48 hours) compared to normal control levels. FIG. 10C shows data for the 11 human miRNA in serum which were identified as having low baseline levels and having altered levels in HIE (0-6 hour, and/or 48 hours) compared to normal control levels. Thus, these combinations or small panels of protein and/or miRNA biomarkers can be used as diagnostic tools for neonatal HIE, for monitoring HIE injury's temporal progression, for predicting MRI-detectable brain injury, and as prognostic tools for 18-24 month outcome (see FIG. 10).

### Example 13. HIE Biomarker Testing

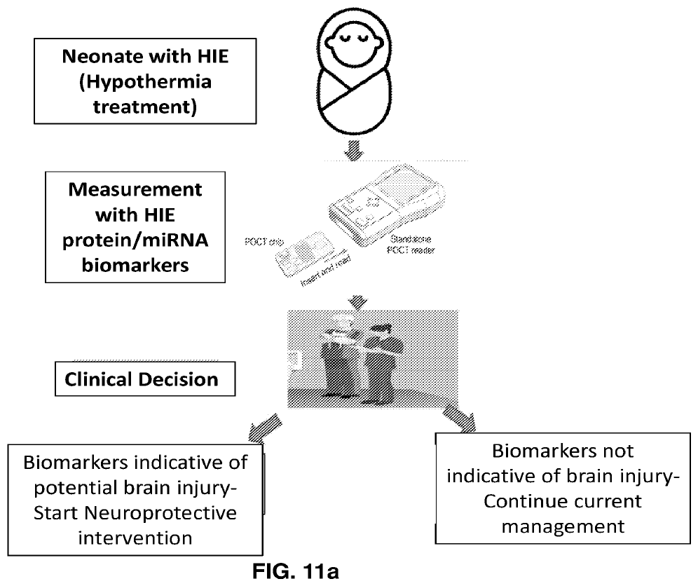

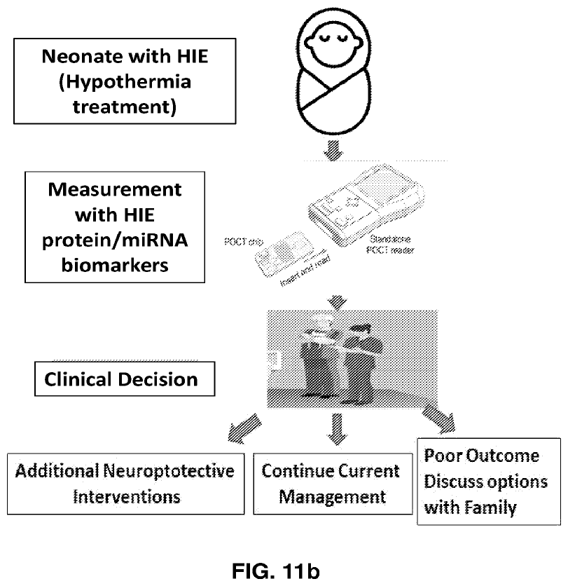

FIG. 11 is a flow chart for an exemplary use of biomarker testing for assistance to clinicians in making a clinical decision for patients. A point-of-care of bedside HIE biomarker test or tests is contemplated as an embodiment of the invention. This test will involve measurement of a combination or small panel of protein and/or miRNA biomarkers as diagnostic and prognostic tools for neonatal HIE, for monitoring HIE injury temporal progression, as a predictor of MRI-detectable brain injury, and as a prognostic tool for the 18- to 24-month outcome of the patient. See FIG. 11.

### Example 14. Clinical Uses

Acute (0-48 hour) UCH-L1 and GFAP serum/plasma/blood concentrations are increased in neonates with HIE compared to control subjects and the increases correlate with the volume of MRI injury. A panel of additional neuroprotein markers (NF-L, Tau) and selected panel of 6 miRNA and their associated temporal profiles can further increase the diagnostic or prognostic properties of UCH-L1 and GFAP alone.

Testing for blood levels of a selected panel of blood-based protein and/or miRNA HIE biomarkers can provide a convenient test to diagnose and determine HIE severity. Prognosis (likelihood of poor versus good cognitive or overall patient outcome) also can be assisted by this method.

### Example 15. Protein Biomarkers as Predictors of Brain Injury in Neonatal Encephalopathy

Patient Demographics and study profile: Correlation of serum biomarkers concentrations with short term outcomes as defined by brain injury on MRI were assessed in 40 patients, and with long term outcomes, as defined by Bayley III neurodevelopmental outcomes, were assessed in a cohort of 20 patients. Patients enrolled in the study were primarily male (65%) with mean gestational age 38.3 weeks (SD±1.9) and mean birth weight 3340 g (SD±783). Patient characteristics at enrollment were analyzed by no/mild and moderate/severe brain injury on MRI. All characteristics were similar between the groups except APGAR score at 5 minutes of life and SARNAT scores (p<0.05). APGAR scores were lower in the moderate/severe brain injury on MRI group, and more infants in the moderate/severe brain injury on MRI group had a stage III initial SARNAT exam. Additional characteristics and details are shown in Table 15.

TABLE 15
Infant Characteristics at Enrollment

| Infant Characteristics | NE (n = 20) | | NE (n = 20) | |
|---|---|---|---|---|
| at Enrollment | No/mild injury on MRI | | Moderate/severe injury on MRI | |
| Sex - no. (%) | | | | |
| Female | 5 | (25) | 9 | (45) |
| Male | 15 | (75) | 11 | (55) |
| Race - no. (%) | | | | |
| White | 11 | (55) | 13 | (65) |
| Black | 5 | (25) | 4 | (20) |
| Other | 4 | (20) | 3 | (15) |
| Gestational Age (weeks), | 38.4 ± 1.6 | | 37.9 ± 2.1 | |
| mean and standard deviation | | | | |
| Birth weight (g), | 3313 ± 747 | | 3358 ± 903 | |
| mean and standard deviation | | | | |
| Apgar score at 1 minute | 2 | (1-3) | 1 | (0.75-2) |
| (median (IQ range) | | | | |
| [symbol] Apgar score at 5 minutes | 5 | (4-6.5) | 3 | (1-4) |
| (median (IQ range)) | | | | |
| Apgar score at 10 minutes | 7 | (5.5-8) | 5 | (3-7) |
| (median (IQ range)) | | | | |
| Sentinel Event - no. (%) | 5 | (25) | 9 | (75) |
| C. Section delivery - no. (%) | 8 | (40) | 12 | (60) |
| History of seizures - no. (%) | 5 | (25) | 11 | (55) |
| [symbol] SARNAT score II - no. (%) | 18 | (90) | 10 | (50) |
| [symbol] SARNAT score III - no. (%) | 2 | (10) | 10 | (50) |

| | | |
|---|---|---|
| Initial pH | 7.08 ± 0.1 | 6.9 ± 0.2 |
| (mean and standard deviation) | | |
| Initial Base Deficit | −16.8 ± 4.8 | −18.4 ± 7.5 |
| (mean and standard deviation) | | |
| Initial Lactate | 10.2 ± 5.7 | 13.1 ± 5.2 |
| (mean and standard deviation) | | |

[symbol]

indicates data missing or illegible when filed

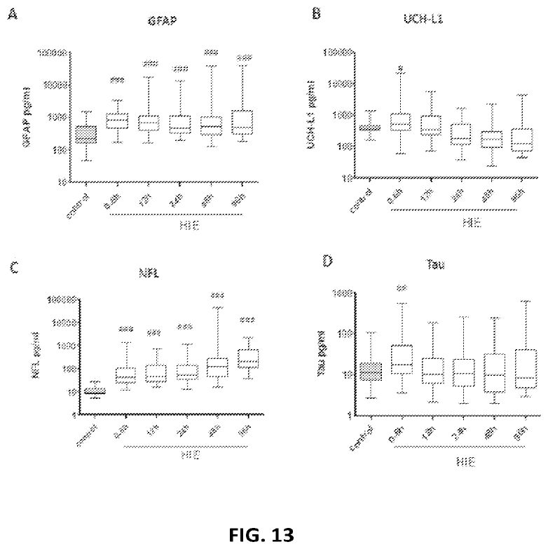

Neuroprotein biomarker time profile in infants with NE compared to controls: From each patient in the NE group, 1 sample at 5 different times was collected as follows: between 0 and 6 hours of life (time 1), 12 hours (time 2), 24 hours (time 3), 48 hours (time 4) and 72 hours of life (time 5). These time points respectively correspond to before, during, and after the hypothermia. The control samples were collected from each patient's umbilical cord at the time of birth. Log-scale median and interquartile range serum concentrations of GFAP. NFL, UCHL-1 and Tau over time are shown in FIG. 13. The serum concentrations of the neuroprotein biomarkers from infants with NE undergoing hypothermia were compared to control subjects. Serum concentrations of GFAP and NFL were increased compared to controls at all time points examined (p<0.05) with GFAP peaking at 0-6 hours and NFL at 96 hours. UCH-L1 (p<0.05) and Tau (p<0.01) serum concentrations in patients with NE undergoing hypothermia were increased at 0-6 hours compared to control samples and were not different from control samples at the other 4 time points examined; 12, 24, 48 and 96 hours.

Biomarker Concentrations and Correlation with MRI Injury: Neonates had an MRI scan at 3 days of life following rewarming. The injury severity score of the basal ganglia (A), watershed (B), basal ganglia/watershed (C) were recorded. For the study, an MRI score less than 3 in any category is considered as no or mild brain injury, while a score above or equal to 3 indicates moderate to severe brain injury to the identified region. Compared to the control serum concentrations of GFAP, serum concentrations in NE infants undergoing hypothermia increased within 6 hours after birth regardless of the MRI injury group in the all three brain regions. In NE infants with moderate/severe brain injury, the GFAP concentrations demonstrated a trend towards increasing over time while the no/mild group demonstrated a trend towards decreasing over time. GFAP serum concentration was increased at 48 and 96 hours of age in neoantes with moderate/severe brain injury on MRI compared to neonates with no/mild injury in all 3 brain regions scored (p<0.05). Similar to GFAP, UCHL1 concentrations in the NE infants undergoing hypothermia regardless of the MRI injury group had a similar trend of peaking at 0-6 hours of life and decreasing over the other time points measured. The no/mild injury group demonstrated a trend towards decreasing more at each time point than the moderate/severe injury group. UCH-L1 serum concentrations were increased in neonates with moderate/severe brain injury compared to no/mild injury at 96 hours of life in all 3 brain regions (p<0.05). NFL concentrations were increased at 96 hours in neonates with moderate/severe injury compared to no/mild injury in the basal ganglia and watershed brain regions (p<0.05). Interestingly, Tau concentrations demonstrated a trend towards decreasing in the no/mild injury group compared to an increase in the moderate/severe group beginning at 48 hours. Tau was increased in the moderate/severe group at 48 and 96 hours in all 3 brain regions (p<0.05). In addition, Tau was increased in the moderate/severe group compared to the no/mild group at 12 hours of age in the watershed brain region (FIG. 13). Receiver operating characteristic (ROC) curves were used to analyze the ability of these biomarkers to detect brain injury severity on MRI. ROC curves were derived for all biomarkers individually as well as a combination of the four markers overtime in patients with no/mild and moderate/severe MRI injury (Table 16). Area under ROC curves (AUC) summarizes diagnostic accuracy, with those approaching 1.00 being very accurate while AUC approaching 0.5 are considered more associated with pure chance. GFAP, UCHL1, NFL and Tau concentrations at all time points showed varying degrees of discrimination (all AUC>0.5). The combination of four markers increased the AUC (blue) compared to the individual AUC and reached a statistical significant difference after 6 hours of life.

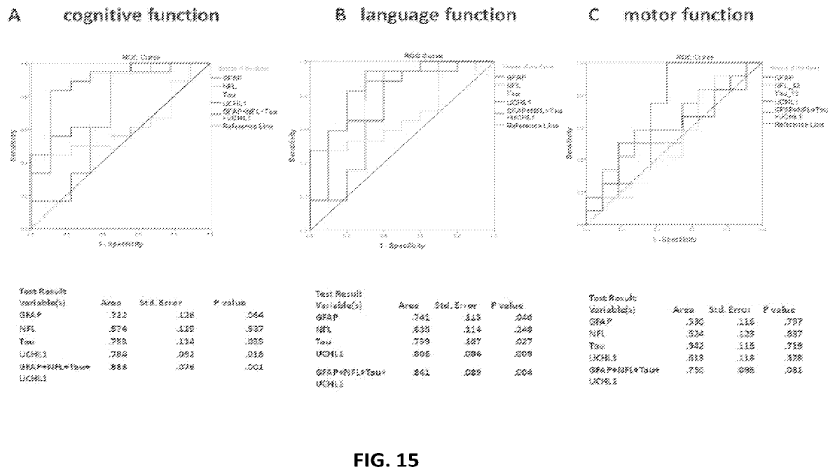

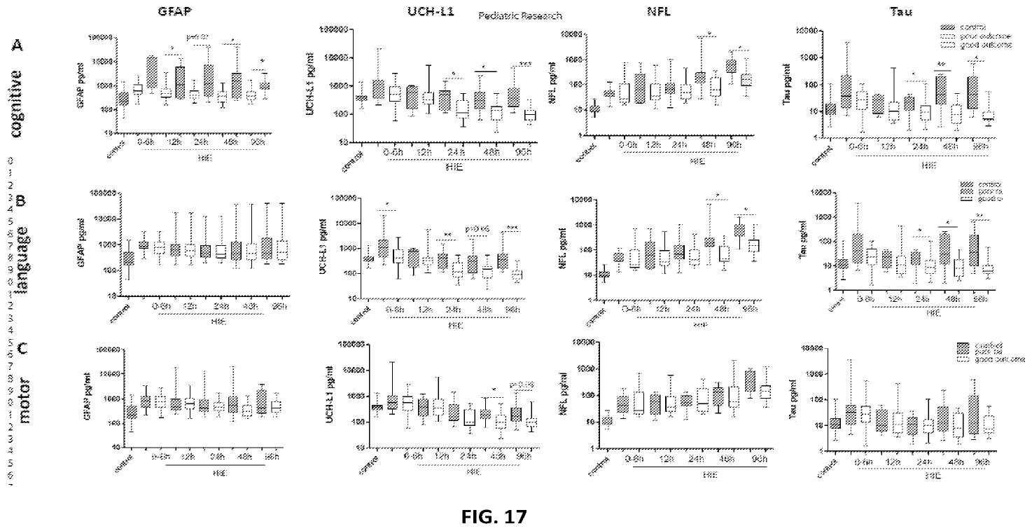

Biomarker concentrations and correlation with neurologic outcomes: We analyzed Bayley III exams (n=20) at 17-24 months of age with an average of 20 months of age. Individual developmental domains on the Bayley III including motor, cognitive and language were analyzed. The 3 developmental domains were classified as a good outcome if the Bayley domain score was 85 or greater or a poor outcome, defined as a Bayley score of less than 85. To determine the ability of these biomarkers to predict the good versus poor developmental outcomes at 17-24 months of age, prognostic models for developmental outcomes ROC were derived for all biomarkers (Table 17). A representative ROC for hour 12 is depicted in FIG. 15. Serum GFAP, NFL, Tau and UCH-L1 levels showed varying degrees of discrimination with a AUC value of 0.772, 0.574, 0.753, 0.784 respectively in cognitive function, 0.741, 0.635, 0.759, 0.806 in language function, 0.53, 0.524, 0.542, 0.613 in motor function between patients with good versus poor outcomes. The combination of the four biomarkers increased the prognostic ability with an AUC 0.883 in cognitive function. AUC 0.841 in language function and AUC 0.75 in motor function. In FIG. 17, log-scale biomarker concentrations were plotted between the good and poor outcomes. GFAP concentrations were elevated at sample collection times of 12, 24, 48 and 96 hours in subjects with poor cognitive outcomes compared to good outcomes at 17-24 (p<0.05). UCH-L1 and Tau concentrations were increased in subjects with poor cognitive outcomes compared to good outcomes at the 24, 48 and 96 hour sampling time points (p<0.05) while NFL was increased in the poor outcome group at 48 and 96 hours (p<0.05). Increased serum concentrations of UCH-L1 at 0-6, 24 and 96 hours (p<0.05), NFL at 48 and 96 hours (p<0.05) and Tau at 24, 48 and 96 hours (p<0.05) were associated with poor outcomes in the language domain. Poor outcomes in the motor domain were associated with increased concentrations of UCH-L1 at 48 hours (p<0.05).

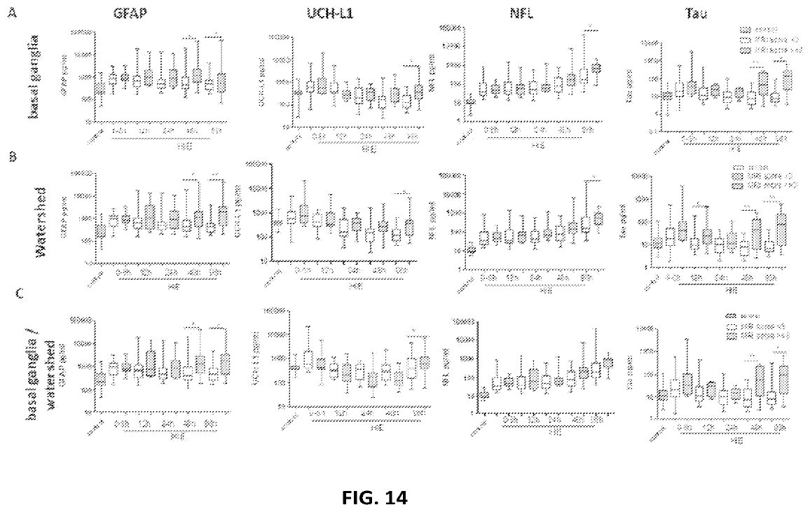

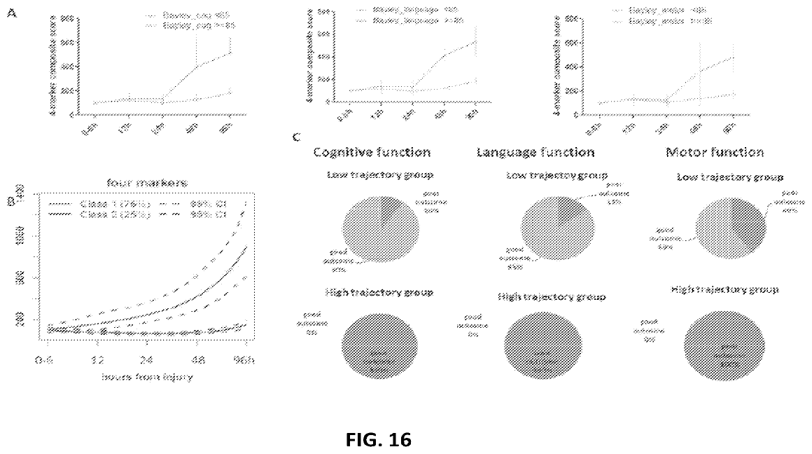

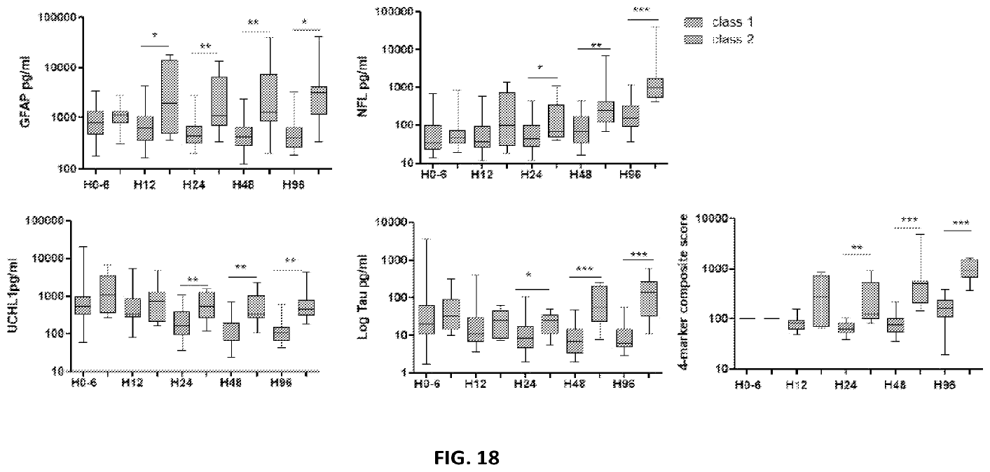

Composite biomarker trajectory analysis of neurologic outcomes: Four-composite biomarker scores were created using relative biomarker concentration changes from baseline (0-6 hours) at each sample collection time point (expressed as a ratio). FIG. 16A shows a 4-marker standardized composite score (GFAP, NF-L, UCHL-1, Tau; mean+SEM) over time for class. For good outcome subjects, the weighted composite score remains slightly negative. In contrast, the four-marker composite score for poor outcome subjects rose sharply upward at 24 hours (p<0.05) and continued to rise over several orders of magnitude. Based on the machine-learning method, two groups with the best fit membership for high trajectory (Class 1, red) and low trajectory (Class 2, blue) marker score data were developed (FIG. 16B). The percent membership for high trajectory class range from 25% for GFAP score to 75% for the four-marker score. Complete concordance of four-marker score trajectory group membership in independently predicting patient outcome is shown in FIG. 14C. At 17-24 months follow-up, all subjects in the high-trajectory group (class 2) had a poor neurologic outcome group, while 90% low-trajectory patients (class 1) belong to the good outcome group. The odds ratio for poor cognitive, language and motor outcome being in the composite high TRAJ class is 2.57 (95% CI: 0.914 to 7.23; z statistics 1.79, significance level at p=0.0435), 2.12 (95% CI: 0.776 to 5.81; z statistics 1.47, significance level at p=0.14) and 0.92 (95% CI: 0.353 to 2.413; z statistics 1.632, significance level at p=0.87) respectively. The two classes' respective biomarker median and interquartile range concentrations over time are shown in FIG. 18. Significant difference in biomarkers concentrations are shown from 24h for GFP, NFL, UCH-L1, Tau and 4-marker composite scores.

### Example 16. Concentration of Serum Biomarkers of Brain Injury in Neonates with a Low Cord pH with or without Mild Hypoxic-Ischemic Encephalopathy

Materials and Methods

A. Patient Population

The University of Florida Institutional Review Board approved all aspects of this study.

1. Low Cord pH with or without Evidence of Mild HIE

In the Neonatal Intensive Care Unit (NICU) at UF Health Gainesville, a sample of blood was obtained from umbilical artery and vein of all inborn neonates. If the neonate's umbilical cord pH was between 7.11-7.15, a Neonatology Fellow or NNP was notified and bedside serial neurologic exams using the modified Sarnat scoring was done every 1-2 hours until 6 hours of life to assess for changes in the neurologic exam. If neonates had worsening neurologic exam, neonates were transitioned to the NICU for evaluation for therapeutic hypothermia. If the cord pH was less than or equal to 7.1, neonates were transitioned to NICU for close monitoring, collection of a sample of blood for analysis (CK, CK-MB, troponin, PT/PTT, fibrinogen, LFT, ABG, lactate) and aEEG monitoring. Regardless of the pH, if the neonate had a normal neurologic exam, normal labs and/or aEEG with no evidence of hypoxic-ischemic injury for the 6-hour monitoring period, the neonate was transitioned back to mother. Neonates that evolved and met the criteria for therapeutic hypothermia during the 6 hours were started on cooling therapy (see below). Neonates with a pH between 7.11-7.15 had clinical labs drawn at the discretion of the attending physician. At time of obtaining the clinical samples, an additional 1 ml of blood in glass gold top was obtained for serum neuroprotein biomarker evaluation. The study team had 24 hours to obtain informed consent for biomarker study. If mother did not consent, the sample was discarded. Neonates who had 1-2 abnormal finding on the modified Sarnat exam met the criteria for mild HIE.

2. Moderate to Severe HIE Neonates

Neonates with HIE who were eligible for hypothermia therapy were enrolled in our biorepository. Entry criteria for hypothermia included a gestational age of 35 weeks or greater, a birth weight of 1.8 kg or greater, and less than or equal to 6 h of age. Enrolled neonates had evidence of encephalopathy as defined by seizures or 3 or more abnormalities on a modified Sarnat exam (56). Evidence of hypoxic-ischemic injury was defined by 1) a pH of 7.0 or less and/or a base deficit of greater than 16, or 2) a pH between 7.01 and 7.15 and/or a base deficit between 10 and 15.9, or 3) no blood gas available and an acute perinatal event (cord prolapsed, heart rate decelerations, uterine rupture) and an APGAR of 5 or less at 5 minutes of life or need for mechanical ventilation at 10 minutes of life (56). In addition, neonates who were transitioned to NICU for a low cord pH of less than or equal to 7.10 who evolved over 6 hours and met criteria for therapeutic hypothermia were also included in this cohort. All neonates undergoing therapeutic hypothermia received a set of labs consisting of CK, CK-MB, troponin, PT/PTT, fibrinogen, liver function test, ABG and lactate at 0-6 hours of life prior to initiation of therapeutic hypothermia. During that period, 1 ml of blood in glass gold top was obtained for neuroprotein biomarkers evaluation.

3. Control Neonates

Control neonates were healthy full-term neonates born at UF Health Gainesville with APGAR scores 8 or more at 5 minutes of life. A single sample of blood was collected from the umbilical cord (artery or vein) at time of birth for assessment of neuroprotein biomarkers.

B. Blood Processing

Serum samples (1 ml) were collected using a 3.5 ml serum separator tube (BD Vacutainer® SST Plus Blood Collection Tube, Franklin Lakes, N.J.). Samples were allowed to clot in an upright position at room temperature for 30 minutes in the processing lab (45±15 minutes from time of collection), then centrifuged at 1200 RCF (g) at room temperature for 15 minutes in a fixed angle centrifuge rotor. Immediately following centrifugation, the serum was transferred from the SST tube using a disposable transfer pipette into a 2 ml cryovial with red cap inserts (USA Scientific, Orlando, Fla.). The serum samples were stored at 4° C. A fiberboard cryogenic storage box (Fisher Part Scientific, Pittsburgh, Pa.) was used to store serum aliquots at −80° C. until analysis for the assays. Blood collection from neonates was done in accordance with common practice as well as state and federal regulations.

C. Biomarker Analysis

1. Enzyme-Linked Immunosorbent Assay (ELISA)

Measurement of GFAP, UCH-L1, NFL and Tau concentrations were measured using the same batch of reagents by investigators blinded to clinical data using SIOMA neuro 4 plex kit in SR-X immunoassay analyzer (Quanterix Corp, Boston, Mass., USA), which runs ultrasensitive paramagnetic bead-based enzyme-linked immunosorbent assays.

D. Statistical Analysis

Data analyses were performed using SAS 9.4. Given the small sample size, nonparametric statistical methods and the Wilcoxon rank-sum test was used.

### Results

A. Patient Population

The demographics of neonates with a low cord pH with or without evidence of mild HIE and moderate-severe HIE were comparable gestational age, birth weight, males and Caesarian section (Table 18). All neonates in the low cord pH group were inborn, whereas 50% of the neonates in moderate-severe HIE group were outborn and transferred for therapeutic hypothermia. The APGAR scores at 1 and 5 min were lower in moderate-severe HIE, 1.89±1.55 and 4.78±1.8, respectively, compared to neonates with low cord pH group, 3.8±3.14 and 7.2±1.8, respectively (Table 18, P<0.05).

Among the 18 neonates in the low cord pH group, 10 neonates had evidence of a low cord pH without evidence of encephalopathy whereas eight neonates had low cord pH with evidence of mild HIE, Sarnat 1. We further compared demographics of the low cord pH neonates without HIE versus mild HIE and found no difference in gestational age, birthweight, and APGAR scores (Table 19). The mean cord pH for the low cord pH group without evidence of HIE was 7.05±0.55 versus 7.07±0.05 in the low pH group with mild HIE. Mean Cord base deficit for the low cord pH group without evidence of HIE was −11.25±3.5 versus −11.41±3.5 in neonates with a low cord pH and mild HIE. Nine of the ten neonates in the low cord pH without evidence of mild HIE had evidence of respiratory acidosis (defined by PaCo2>60) in cord blood whereas 7 out of 8 neonates in the low pH group with mild HIE had evidence of respiratory acidosis with mean PaCo2 of 84.2±11.3 and 74.66±16.5 respectively.

B. Serum Concentration of Biomarkers in Neonates with a Low Cord pH Compared to Control Neonates and Neonates with Moderate to Severe HIE

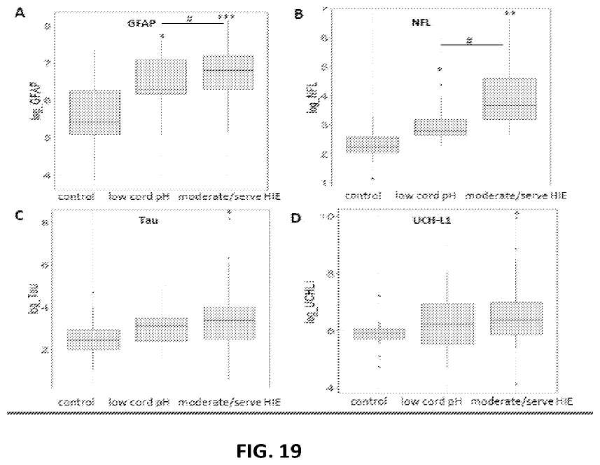

Serum concentration of a panel of biomarkers were measured from each cohort from serum samples obtained at 0-6 hours of life (FIG. 19). The serum concentration of GFAP was increased in neonates with moderate to severe HIE compared to neonates with a low cord pH (FIG. 19A)(p<0.05). GFAP concentrations were increased in neonates with a low cord pH compared to control neonates (p<0.05)(FIG. 19A). NFL concentrations were elevated in neonates with moderate to severe HIE compared to neonates with a low cord pH and control neonates (p<0.05)(FIG. 19C). UCH-L1 and Tau concentrations were increased in neonates with moderate to severe HIE compared to control neonates (p<0.05)(FIG. 19B,C).

C. Serum Concentration of Biomarkers in Neonates with Mild HIE and Neonates with Moderate to Severe HIE

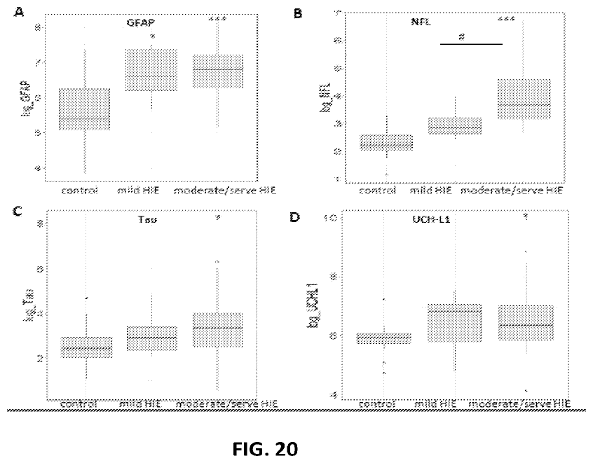

The serum concentration of the four biomarkers was compared between the control neonates, neonates with mild HIE and those with moderate to severe HIE. The serum concentrations of GFAP and NFL were higher in the mild HIE group compared with controls (p<0.05) but lower than neonates with moderate to severe HIE (p<0.05)(FIG. 20A, B). Serum concentrations of all four biomarkers, GFAP, NFL, Tau and UCH-L1 were higher in neonates with moderate to severe HIE compared to the control group (p<0.05)(FIG. 20 A-D).

D. Serum Biomarker Panel Concentration in Neonates with a Low Cord pH Based on pH, Lactate, Base Deficit and Sentinel Events

Eighteen neonates with low cord pH with or without mild HIE were further analyzed using physiologic parameters such as pH, lactate, base deficit and the presence of sentinel events to examine if any of these parameters were correlated with the concentration of the four neuroprotein biomarker panel. The cohort of 18 neonates was divided into 2 groups for each of these parameters: a pH less than or equal to 7 (n=3) compared to a pH of 7 or higher (n=15), a serum lactate concentration of greater than or equal to 7 (n=5) compared to less than 7 (n=13), a base deficit equal to or greater than 15 (n=2) versus less than 15 (n=16), and neonates with sentinel events (n=3) compared to neonates without sentinel events (n=15). The pH was chosen since a pH less than 7 is associated with a higher risk of long-term neurologic deficits, a lactate greater than 7 is associated with a higher risk of encephalopathy and a base deficit greater than 15 a more severe metabolic acidosis (57-60). The presence of a sentinel event allowed for some understanding of the timing of the rise in the biomarkers. Sentinel events were defined as umbilical cord mishap (cord prolapsed), uterine rupture, placental abruption, shoulder dystocia and major maternal hemorrhage, trauma, cardiorespiratory arrest or seizures immediately preceding delivery (61).

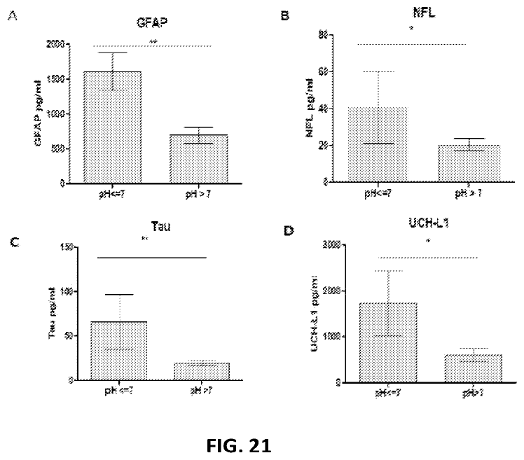

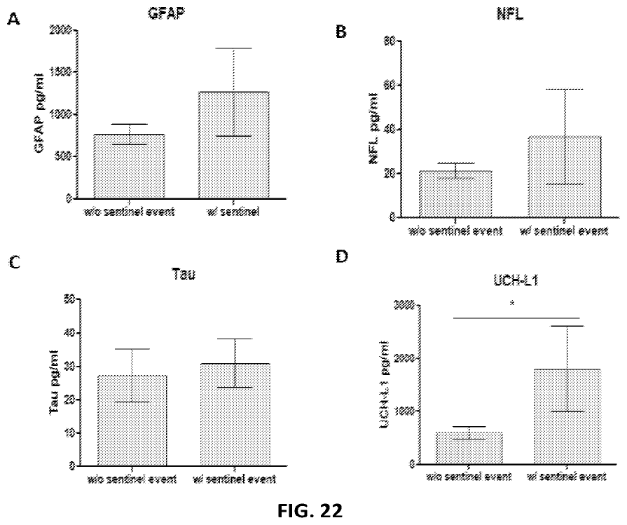

The serum concentration of GFAP, NFL, Tau and UCH-L1 were higher in neonates with a pH less than or equal to 7 compared to neonates with a pH higher than 7 (p<0.05)(FIG. 21). NFL and UCH-L1 concentrations were higher in neonates with a base deficit of 15 or greater (p<0.05). Lactate concentrations of 7 or higher were associated with higher serum concentrations of NFL and UCH-L1 (p<0.05). Neonates with a known sentinel event had higher serum concentrations of UCH-L1 (p<0.05)(FIG. 22).

E. Serum Concentration of Biomarker Panel in Neonates with a Low Cord pH with Mild HIE Compared to a Low Cord pH without Mild HIE

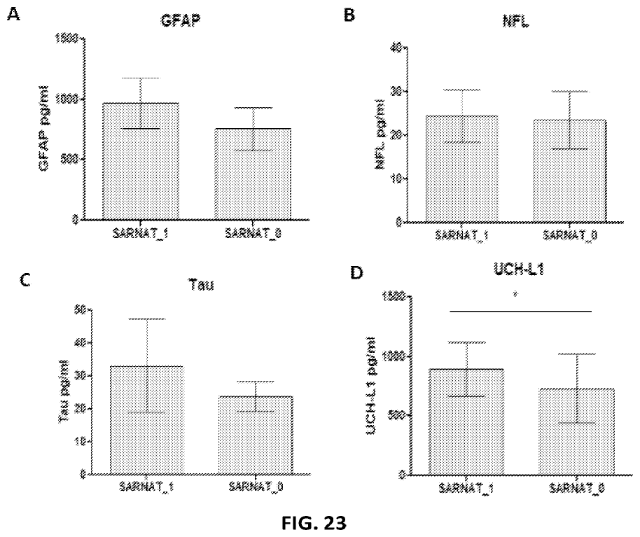

The cohort of 18 neonates was divided into two groups, those with a normal neurologic exam (n=10), Sarnat 0, and those with a Sarnat 1 exam (n=8, mild HIE). UCH-L1 serum concentrations were increased in neonates with mild HIE compared to those without mild HIE (p<0.05)(FIG. 23).

TABLE 18
Demographic data

| | Low cord | Moderate/severe | |
|---|---|---|---|
| | pH (n = 18) | HIE (N = 40) | p-value |
| Gestational age | 38.7 + −1.46 | 38.22 + −1.87 | 0.2 |
| Gender (Male) | 12 (67%) | 27 (67%) | |
| Birth weight (grams) | 3419 ± 516 | 3367 ± 825 | 0.77 |
| Outborn | 0 | 35% (14) | |
| Mode of delivery | 9 (50%) | 20 (50%) | 1.0 |
| C section | | | |
| APGAR @1 and | 3.8 ± 3.14 | 1.89 ± 1.55 | 0.02 |
| 5 min | 7.2 ± 1.8 | 4.78 ± 1.8 | <0.001 |

TABLE 19

| | Low Cord pH without | Low cord pH with mild | |
|---|---|---|---|
| | HIE (n = 10) | HIE (n = 8) | p-value |
| Gestational age | 38.4 ± 0.96 | 39.12 ± 1.9 | 0.36 |
| Birth weight | 3338 ± 543 | 3521 ± 495 | 0.46 |
| Cord pH | 7.05 ± 0.55 | 7.07 ± 0.05 | 0.33 |
| Cord base | −11.25 ± 3.5 | −11.41 ± 3.5 | 0.92 |
| deficit | | | |
| Cord PaCo2 | 84.2 ± 11.3 | 74.66 ± 16.5 | 0.18 |
| APGARS at 1 min | 4.3 ± .6 | 3.25 ± 2.4 | 0.49 |
| At 5 min | 7.3 ± 2.11 | 7.12 ± 1.45 | 0.83 |

### REFERENCES

All references listed below and throughout the specification are hereby incorporated by reference in their entirety.
1. Di Pietro et al., Salivary MicroRNAs: Diagnostic Markers of Mild Traumatic Brain Injury in Contact-Sport. Front. Mol. Neurosci., 11:290, 2018.
2. Douglas-Escobar et al., Weiss, M D, A Pilot Study of Novel biomarkers in neonates with Hypoxic-Ischemic Encephalopathy. Pediatr. Res. 68(6):531-536, 2010.
3. Douglas-Escobar et al. and Weiss, UCH-L1 and GFAP Serum Levels in Neonates with Hypoxic-Ischemic Encephalopathy: A Single Center Pilot Study. Front. Neurol. 5:273, 2014.
4. Hongwei et al., HIF1α and HIF2α mediated UCH-L1 upregulation in hypoxia-induced neuronal injury following neuronal hypoxic ischemic encephalopathy. Int. J. Clin. Exp. Pathol., 9(2):2677-2685, 2016.
5. Ennen et al., Glial fibrillary acidic protein as a biomarker for neonatal hypoxic-ischemic encephalopathy treated with whole-body cooling. Am. J. Obstet. Gynecol., 205(3):251 el-7, 2011.
6. Blennow et al., Brain-specific proteins in the cerebrospinal fluid of severely asphyxiated newborn infants. Acta Paediatr., 90(10):1171-1175, 2001.
7. Looney et al., Glial Fibrillary Acidic Protein Is Not an Early Marker of Injury in Perinatal Asphyxia and Hypoxic-Ischemic Encephalopathy. Front. Neurol., 6:264, 2015.
8. Korley et al., Performance Evaluation of a Multiplex Assay for Simultaneous Detection of Four Clinically Relevant TBI Biomarkers. J. Neurotrauma, July 23. doi: 10.1089/neu.2017.5623.2018.
9. Azzopardi et al., Moderate hypothermia to treat perinatal asphyxial encephalopathy. N. Engl. J. Med. 361(14):1349-58, 2009.
10. Badawi et al., Intrapartum risk factors for newborn encephalopathy: the Western Australian case-control study. B.M.J. 317(7172):1554-1558, 1998.
11. Bembea et al., Glial fibrillary acidic protein as a brain injury biomarker in children undergoing extracorporeal membrane oxygenation. Pediatr. Crit. Care Med. 12(5):572-579, 2010.
12. Boichot et al., Term neonate prognoses after perinatal asphyxia: contributions of MR imaging, MR spectroscopy, relaxation times, and apparent diffusion coefficients. Radiology. 239(3):839-848, 2006.
13. Bonifacio et al., A new neurological focus in neonatal intensive care. Nat. Rev. Neurol. 7(9):485-494, 2011.
14. Brophy et al., Biokinetic analysis of ubiquitin C-terminal hydrolase-L1 (UCH-L1) in severe traumatic brain injury patient biofluids. J. Neurotrauma. 28(6):861-870, 2011.
15. Chalak et al., Biomarkers for severity of neonatal hypoxic-ischemic encephalopathy and outcomes in newborns receiving hypothermia therapy. J. Pediatr. 164(3):468-474 el, 2014.
16. Czeiter et al., Brain injury biomarkers may improve the predictive power of the IMPACT outcome calculator. J. Neurotrauma. 29(9):1770-1778, 2012.
17. Day and Thompson, UCH-L1 (PGP 9.5): neuronal biomarker and ubiquitin system protein. Prog. Neurobiol. 90(3):327-362, 2009.
18. Douglas-Escobar and Weiss, Hypoxic-ischemic encephalopathy: a review for the clinician. J.A.M.A. Pediatr. 169(4):397-403, 2015.
19. Eng, Glial fibrillary acidic protein (GFAP): the major protein of glial intermediate filaments in differentiated astrocytes. J. Neuroimmunol. 8(4-6):203-214, 1985.
20. Finer et al., Hypoxic-ischemic encephalopathy in term neonates: perinatal factors and outcome. J. Pediatr. 98(1):112-117, 1984.
21. Fraser et al., Severe traumatic brain injury in children elevates glial fibrillary acidic protein in cerebrospinal fluid and serum. Pediatr. Crit. Care Med. 12(3):319-324, 2010.
22. Garfinkle et al., Cooling in the real world: Therapeutic hypothermia in hypoxic-ischemic encephalopathy. Eur. J. Paediatr. Neurol. 2013.
23. Gazzolo et al., Neurological abnormalities in full-term asphyxiated newborns and salivary S100B testing: the “Cooperative Multitask against Brain Injury of Neonates” (CoMBINe) international study. PLoS One. 10(1):e0115194, 2015.
24. Gunn et al., Therapeutic hypothermia changes the prognostic value of clinical evaluation of neonatal encephalopathy. J, Pediatr, 152(1):55-58, 2008.
25. Hayes et al., Glial fibrillary acidic protein: a promising biomarker in pediatric brain injury. Pediatr. Crit. Care Med. 12(5):603-604, 2011.
26. Jouvet et al., Reproducibility and accuracy of MR imaging of the brain after severe birth asphyxia. A.J.N.R. Am. J. Neuroradiol. 20(7):1343-1348, 1999.
27. Kurinczuk et al., Epidemiology of neonatal encephalopathy and hypoxic-ischaemic encephalopathy. Early Hum. Dev. 86(6):329-38, 2010.
28. Leijser et al., Prediction of short-term neurological outcome in full-term neonates with hypoxic-ischaemic encephalopathy based on combined use of electroencephalogram and neuro-imaging. Neuropediatrics. 38(5):219-227, 2007.
29. Li, et al., MiRNA-210 induces microglial activation and regulates microglia-mediated neuroinflammation in neonatal hypoxic-ischemic encephalopathy. Cell. Mol. Immunol. 2019.
30. Liu et al., Ubiquitin C-terminal hydrolase-L1 as a biomarker for ischemic and traumatic brain injury in rats. Eur. J. Neurosci. 31(4):722-732, 2010.
31. Looney et al., Altered Expression of Umbilical Cord Blood Levels of miR-181b and Its Downstream Target mUCH-L1 in Infants with Moderate and Severe Neonatal Hypoxic-Ischaemic Encephalopathy. Mol. Neurobiol. 2018.
32. Ma et al., MicroRNA-210 Suppresses Junction Proteins and Disrupts Blood-Brain Barrier Integrity in Neonatal Rat Hypoxic-Ischemic Brain Injury. Int. J. Mol. Sci. 18(7), 2017.
33. Massaro et al., Plasma Biomarkers of Brain Injury in Neonatal Hypoxic-Ischemic Encephalopathy. J. Pediatr. 194:67-75 el, 2018.
34. Massaro et al., Serum biomarkers of MRI brain injury in neonatal hypoxic ischemic encephalopathy treated with whole-body hypothermia: a pilot study. Pediatr. Crit. Care Med. 14(3):310-317, 2013.
35. Mondello et al., Blood-based diagnostics of traumatic brain injuries. Expert Rev. Mol. Diagn. 11(1):65-78, 2010.
36. Mondello et al., Glial neuronal ratio: a novel index for differentiating injury type in patients with severe traumatic brain injury. J. Neurotrauma. 29(6):1096-1104, 2011.
37. Mondello et al., Clinical utility of serum levels of ubiquitin C-terminal hydrolase as a biomarker for severe traumatic brain injury. Neurosurgery. 70(3):666-675, 2011.
38. Murray, Biomarkers in neonatal hypoxic-ischemic encephalopathy-Review of the literature to date and future directions for research. Handbook Clin. Neurol. 162:281-293, 2019.
39. Nelson et al., Antecedents of neonatal encephalopathy in the Vermont Oxford Network Encephalopathy Registry. Pediatrics. 130(5):878-886, 2012.
40. Oe et al., Regulatory non-coding RNAs in nervous system development and disease. Front. Biosci. (Landmark Ed.). 24:1203-1240, 2019.
41. O'Sullivan et al., Validation of Altered Umbilical Cord Blood MicroRNA Expression in Neonatal Hypoxic-Ischemic Encephalopathy. J.A.M.A. Neurol. 76(3):333-341, 2019.
42. Papa et al., Ubiquitin C-terminal hydrolase is a novel biomarker in humans for severe traumatic brain injury. Crit. Care Med. 38(1):138-144, 2009.
43. Papa et al., Elevated levels of serum glial fibrillary acidic protein breakdown products in mild and moderate traumatic brain injury are associated with intracranial lesions and neurosurgical intervention. Ann. Emerg. Med. 59(6):471-483, 2011.
44. Papa et al., Serum levels of ubiquitin C-terminal hydrolase distinguish mild traumatic brain injury from trauma controls and are elevated in mild and moderate traumatic brain injury patients with intracranial lesions and neurosurgical intervention. J. Trauma Acute Care Surg. 72(5):1335-1344, 2012.
45. Ponnusamy and Yip, The role of microRNAs in newborn brain development and hypoxic ischaemic encephalopathy. Neuropharmacology. 149:55-65, 2019.
46. Qiu et al., Neuroprotective effects of microRNA-210 on hypoxic-ischemic encephalopathy. Biomed. Res. Int. 2013:350419, 2013.
47. Robertson et al., School performance of survivors of neonatal encephalopathy associated with birth asphyxia at term. J. Pediatr. 114(5): p. 753-760, 1989.
48. Sarnat and Sarnat, Neonatal encephalopathy following fetal distress. A clinical and electroencephalographic study. Arch. Neurol. 33(10):696-705, 1976.
49. Tagin et al., Hypothermia for neonatal hypoxic ischemic encephalopathy: an updated systematic review and meta-analysis. Arch. Pediatr. Adolesc. Med. 166(6):558-66, 2012.
50. Thoresen et al., Effect of hypothermia on amplitude-integrated electroencephalogram in infants with asphyxia. Pediatrics. 126(1):e131-e139, 2010.
51. van Laerhoven et al., Prognostic tests in term neonates with hypoxic-ischemic encephalopathy: a systematic review. Pediatrics. 131(1):88-98, 2012.
52. Volpe, Neurology of The Newborn. 2001, Philadelphia: W.B. Saunders.
53. Wang et al., Combined prediction of miR-210 and miR-374a for severity and prognosis of hypoxic-ischemic encephalopathy. Brain Behav. 8(1):e00835, 2018.
54. Wyatt et al., Determinants of outcomes after head cooling for neonatal encephalopathy. Pediatrics. 119(5):912-21, 2007.
55. Yoshizawa et al., Salivary biomarkers: toward future clinical and diagnostic utilities. Clin. Microbiol. Rev. 26(4):781-791, 2013.
56. Chalak L F, Nguyen K A, Prempunpong C, Heyne R, Thayyil S, Shankaran S, et al. Prospective research in infants with mild encephalopathy identified in the first six hours of life: neurodevelopmental outcomes at 18-22 months. Pediatr Res. 2018; 84(6):861-8.
57. MacLennan A. A template for defining a causal relation between acute intrapartum events and cerebral palsy: international consensus statement. BMJ. 1999; 319(7216):1054-9.
58. Kelly R, Ramaiah S M, Sheridan H, Cruickshank H. Rudnicka M, Kissack C, et al. Dose-dependent relationship between acidosis at birth and likelihood of death or cerebral palsy. Arch Dis Child Fetal Neonatal Ed. 2018; 103(6):F567-F72.
59. Shah S, Tracy M, Smyth J. Postnatal lactate as an early predictor of short-term outcome after intrapartum asphyxia. J Perinatol. 2004; 24(1):16-20.
60. Low J A, Lindsay B G, Derrick E J. Threshold of metabolic acidosis associated with newborn complications. Am J Obstet Gynecol. 1997; 177(6):1391-4.
61. Bonifacio S L, Glass H C, Vanderpluym J, Agrawal A T, Xu D, Barkovich A J, et al. Perinatal events and early magnetic resonance imaging in therapeutic hypothermia. J Pediatr. 2011; 158(3):360-5.

1. A method of diagnosing neonatal encephalopathy (NE) in a patient in need thereof, comprising:
(a) obtaining at least one blood, plasma, serum, or CSF sample from the patient;
(b) detecting the presence and the amount of NE biomarkers GFAP and UCH-L1 in the sample compared to normal control level;
(c) determining that the patient has suffered an ischemic event if the amount of the at least one NE biomarker is elevated relative to the control level; and
(d) optionally, administering hypothermic treatment to the patient if an ischemic event is determined.

2. The method of claim 1, wherein the at least one NE biomarker tested in step (b) further comprises NF-L or Tau, or both.

3. The method of claim 1, wherein the at least one NE biomarker tested in step (b) further comprises NF-L or Tau, in combination with GFAP and/or UCH-L1.

4. The method of claim 1, further comprising detecting in the sample one or more additional biomarkers compared to normal control sample, the one or more additional biomarkers comprising one or more miRNAs enumerated in Table 1.

5. The method of claim 4, wherein the one or more additional biomarkers comprise hsa-mir-145-5p, hsa-mir-16-5p, hsa-mir-15a-5p, hsa-mir-17-5p, hsa-let-7g-5p, hsa-mir-214-3p, hsa-mir-338-3p, hsa-mir-132-3p, hsa-mir-23a-3p, hsa-mir-26b-5p, or hsa-mir-146a-5p, or a combination thereof.

6. The method of claim 2, further comprising testing the sample for one or more additional miRNA biomarkers that reflect NE/encephalopathy status selected from the group consisting of hsa-mir-145-5p, hsa-mir-16-5p, hsa-mir-15a-5p, hsa-mir-17-5p, hsa-let-7g-5p, hsa-mir-214-3p, hsa-mir-338-3p, hsa-mir-132-3p, hsa-mir-23a-3p, hsa-mir-26b-5p and hsa-mir-146a-5p.

7. The method of claim 1, wherein the sample is obtained at 0-6 hours after birth.

8. The method of claim 7, further comprising obtaining additional samples at times selected from one or more of 24 hours, 48 hours, 96 hours, and 120 hours.

9. A method of determining the severity of NE in a patient in need thereof, comprising:
(a) obtaining at least one blood, plasma, serum, or CSF sample from the patient;
(b) detecting an amount of NE biomarkers GFAP and UCH-L1 compared to normal control level;
(c) determining the severity of NE based on a deviation of the NE biomarker amount with respect to control level;
(d) prognosticating NE patient outcome;
(e) determining if the NE patients should be treated with therapies selected from the group consisting of hypothermia and brain cooling; and
(f) determining if the NE patients on therapies such as hypothermia/brain cooling are responding to the treatment.

10. The method of claim 9, further comprising testing the sample for one or more additional biomarkers selected from the group consisting of NF-L, Tau.

11. The method of claim 9 or 10, further comprising detecting an amount of one or more miRNAs enumerated in Table 1, optionally, wherein the miRNAs are one or more selected from the group consisting of hsa-mir-145-5p, hsa-mir-16-5p, hsa-mir-15a-5p, hsa-mir-17-5p, hsa-let-7g-5p, hsa-mir-214-3p, hsa-mir-338-3p, hsa-mir-132-3p, hsa-mir-23a-3p, hsa-mir-26b-5p and hsa-mir-146a-5p.

12. The method of claim 9, wherein the sample is obtained at 0-6 hours after birth.

13. The method of claim 11, further comprising obtaining additional samples at times selected from one or more of 24 hours, 48 hours, 96 hours, and 120 hours.

14. A method of determining the prognosis of an NE patient in need thereof, comprising:
(a) obtaining at least one blood, plasma, serum, or CSF sample from the patient;
(b) testing the sample for the presence and the amount of NE biomarkers 1-6 times between 0 hours and 96 hours after birth, wherein the NE biomarkers comprise GFAP or UCH-L1, or both;
(c) determining that the patient will respond to hypothermic treatment based on the amounts of the NE biomarkers; and
(d) administering hypothermic treatment to the patient if it is determined that the patient will respond.

15. The method of claim 14, further comprising testing the sample for one or more additional biomarkers selected from the group consisting of NF-L, Tau.

16. The method of claim 14 or 15, further comprising detecting an amount of one or more additional biomarkers enumerated in Table 1 compared to a normal control level.

17. The method of claim 16, wherein the one or more additional biomarkers comprise hsa-mir-145-5p, hsa-mir-16-5p, hsa-mir-15a-5p, hsa-mir-17-5p, hsa-let-7g-5p, hsa-mir-214-3p, hsa-mir-338-3p, hsa-mir-132-3p, hsa-mir-23a-3p, hsa-mir-26b-5p, or hsa-mir-146a-5p, or a combination thereof.

18. The method of any of claims 1-17, wherein the sample is obtained at 0-6 hours after birth.

19. The method of claim 18, further comprising obtaining additional samples at times selected from one or more of 24 hours, 48 hours, 96 hours, and 120 hours.

20. A method determining the responder effectiveness status for hypothermic treatment of an NE patient in need thereof, comprising:
(a) obtaining at least one blood, plasma, serum, or CSF sample from the patient;
(b) testing the sample for the presence and the amount of one or more NE biomarkers selected from the group consisting of GFAP, UCH-L1, NF-L, Tau, hsa-mir-145-5p, hsa-mir-16-5p, hsa-mir-15a-5p, hsa-mir-17-5p, hsa-let-7g-5p, hsa-mir-214-3p, hsa-mir-338-3p, hsa-mir-132-3p, hsa-mir-23a-3p, hsa-mir-26b-5p and hsa-mir-146a-5p compared to normal control sample;
(c) Combination of at least two or more of hsa-mir-145-5p, hsa-mir-16-5p, hsa-mir-15a-5p, hsa-mir-17-5p, hsa-let-7g-5p, hsa-mir-214-3p, hsa-mir-338-3p, hsa-mir-132-3p, hsa-mir-23a-3p, hsa-mir-26b-5p and hsa-mir-146a-5p; and
(d) Combination of at least two or more miRNA biomarkers listed in Table 1 with at least one or more protein biomarker selected from GFAP, UCH-L1, Tau and NF-L).

21. A kit comprising tools, reagents and equipment that enable immunoassay (such as antibodies, aptamers) and/or miRNA amplification (e.g. PCR, RCA) based detection of 1 or more protein biomarkers, or more miRNA biomarkers or combined one or more protein biomarker with one or more miRNA biomarker at either point of care (POC) or core lab test setting using biofluids from NE patient at one or more time points.
